(19) United States
(12) Patent Application Publication
(10) Pub. No.: US 2025/0104826 A1
(43) Pub. Date: Mar. 27, 2025

(54) SYSTEMS AND METHODS FOR MONITORING THE USE OF A RESPIRATORY THERAPY SYSTEM BY AN INDIVIDUAL WITH DIABETES

(71) Applicant: RESMED DIGITAL HEALTH INC., San Diego, CA (US)

(72) Inventors: Redmond SHOULDICE, Dublin (IE); Graeme Alexander LYON, Dublin (IE); Ehsan CHAH, Dublin (IE)

(21) Appl. No.: 18/725,041

(22) PCT Filed: Dec. 28, 2022
(86) PCT No.: PCT/IB2022/062820

§ 371 Date: Jun. 27, 2024

Related U.S. Application Data
(60) Provisional application No. 63/295,259, filed on Dec. 30, 2021.

(51) Int. Cl. G16H 20/10; G16H 20/30; G16H 20/60

(52) U.S. Cl. CPC G16H 20/10; G16H 20/30; G16H 20/60

(57) ABSTRACT

A method comprises receiving data associated with a diabetes treatment plan of the individual. The method further comprises receiving data associated with a respiratory therapy plan of the individual. The respiratory therapy plan is implementable by a respiratory therapy system during a sleep session. The method further comprises determining a potential interaction between the diabetes treatment plan of the individual and the respiratory therapy plan of the individual. The method further comprises, based on the interaction, updating the diabetes treatment plan of the individual.

### CROSS-REFERENCE TO RELATED APPLICATIONS

This application claims the benefit of, and priority to, U.S. Provisional Patent Application No. 63/295,259 filed on Dec. 30, 2021, which is hereby incorporated by reference herein in its entirety.

### TECHNICAL FIELD

The present disclosure relates generally to systems and methods for monitoring an individual with diabetes, and more particularly, to systems and methods for determining interactions between a diabetes treatment plan and a respiratory therapy plan and/or mitigating or optimizing those interactions.

### BACKGROUND

Many individuals suffer from sleep-related and/or respiratory-related disorders such as, for example, Sleep-Disordered Breathing (SDB), which can include Obstructive Sleep Apnea (OSA), Central Sleep Apnea (CSA), other types of apneas such as mixed apneas and hypopneas, Respiratory Effort Related Arousal (RERA), and snoring. These individuals may also suffer from other health conditions (which may be referred to as comorbidities), such as insomnia (e.g., difficulty in initiating sleep, frequent or prolonged awakenings after initially falling asleep, and/or an early awakening with an inability to return to sleep), Periodic Limb Movement Disorder (PLMD), Restless Leg Syndrome (RLS), Cheyne-Stokes Respiration (CSR), respiratory insufficiency, Obesity Hyperventilation Syndrome (OHS), Chronic Obstructive Pulmonary Disease (COPD), Neuromuscular Disease (NMD), rapid eye movement (REM) behavior disorder (also referred to as RBD), dream enactment behavior (DEB), hypertension, diabetes, stroke, and chest wall disorders. These individuals are often treated using a respiratory therapy system (e.g., a continuous positive airway pressure (CPAP) system), which delivers pressurized air to aid in preventing the individual's airway from narrowing or collapsing during sleep. Individuals with diabetes who use a respiratory therapy system are often impacted, positively or negatively, by the use of the respiratory therapy system. For example, the use of the respiratory therapy system can impact the effectiveness of an individual's diabetes treatment plan. Thus, it would be beneficial to be able to monitor an individual with diabetes who uses a respiratory therapy system, and adjust the diabetes treatment plan or the use of the respiratory therapy system as needed. The present disclosure is directed to solving this and other problems.

### SUMMARY

According to some implementations of the present disclosure, a method comprises receiving data associated with a diabetes treatment plan of the individual; receiving data associated with a respiratory therapy plan of the individual, the respiratory therapy plan being implementable by a respiratory therapy system during a sleep session; determining a potential interaction between the diabetes treatment plan of the individual and the respiratory therapy plan of the individual; and based on the interaction, updating the diabetes treatment plan of the individual.

According to some implementations of the present disclosure, a system comprises a respiratory therapy system, a memory device, and a control system. The respiratory therapy system includes a respiratory therapy device configured to supply pressurized air, and a user interface coupled to the respiratory therapy device via a conduit. The user interface is configured to engage a user and aid in directing the supplied pressurized air to an airway of the user. The memory device stores machine-readable instructions. The control system is coupled to the memory device, and includes one or more processors configured to execute the machine-readable instructions to implement a method. The method comprises receiving data associated with a diabetes treatment plan of the individual; receiving data associated with a respiratory therapy plan of the individual, the respiratory therapy plan being implementable by a respiratory therapy system during a sleep session; determining a potential interaction between the diabetes treatment plan of the individual and the respiratory therapy plan of the individual; and based on the interaction, updating the diabetes treatment plan of the individual.

According to some implementations of the present disclosure, a method comprises receiving blood glucose data indicative of one or more blood glucose measurements of the individual; receiving sleep data of the individual that is associated with use of a respiratory therapy system by the individual during one or more prior sleep sessions; based at least in part on the received data, adjusting a diabetes treatment plan of the individual, adjusting one or more settings of the respiratory therapy system, or both.

According to some implementations of the present disclosure, a system comprises a respiratory therapy system, a memory device, and a control system. The respiratory therapy system includes a respiratory therapy device configured to supply pressurized air, and a user interface coupled to the respiratory therapy device via a conduit. The user interface is configured to engage a user and aid in directing the supplied pressurized air to an airway of the user. The memory device stores machine-readable instructions. The control system is coupled to the memory device, and includes one or more processors configured to execute the machine-readable instructions to implement a method. The method comprises receiving blood glucose data indicative of one or more blood glucose measurements of the individual; receiving sleep data of the individual that is associated with use of a respiratory therapy system by the individual during one or more prior sleep sessions; based at least in part on the received data, adjusting a diabetes treatment plan of the individual, adjusting one or more settings of the respiratory therapy system, or both.

According to some implementations of the present disclosure, a method comprises receiving blood glucose data indicative of one or more blood glucose measurements of the individual during a sleep session; receiving sleep data associated with the individual during the sleep session; and based at least in part on the received data, causing an action to be performed.

According to some implementations of the present disclosure, a system comprises a respiratory therapy system, a memory device, and a control system. The respiratory therapy system includes a respiratory therapy device configured to supply pressurized air, and a user interface coupled to the respiratory therapy device via a conduit. The user interface is configured to engage a user and aid in directing the supplied pressurized air to an airway of the user. The memory device stores machine-readable instructions. The control system is coupled to the memory device, and includes one or more processors configured to execute the machine-readable instructions to implement a method. The method comprises receiving blood glucose data indicative of one or more blood glucose measurements of the individual during a sleep session; receiving sleep data associated with the individual during the sleep session; and based at least in part on the received data, causing an action to be performed.

The above summary is not intended to represent each embodiment or every aspect of the present invention. Additional features and benefits of the present invention are apparent from the detailed description and figures set forth below.

### BRIEF DESCRIPTION OF THE DRAWINGS

FIG. 1 is a functional block diagram of a system for monitoring a user with diabetes, according to some implementations of the present disclosure;

FIG. 2 is a perspective view of at least a portion of the system of FIG. 1, a user of the system, and a bed partner, according to some implementations of the present disclosure;

FIG. 3 illustrates an exemplary timeline for a sleep session, according to some implementations of the present disclosure;

FIG. 4 illustrates an exemplary hypnogram associated with the sleep session of FIG. 3, according to some implementations of the present disclosure;

FIG. 5A is a plot of respiratory events and blood glucose levels over time in an individual with controlled blood glucose levels;

FIG. 5B is a plot of respiratory events and blood glucose levels over time in an individual with less controlled blood glucose levels as compared to the individual of FIG. 5A;

FIG. 6 is a flow diagram of a first method for monitoring an individual with diabetes, according to some implementations of the present disclosure;

FIG. 7 is a flow diagram of a second method for monitoring an individual with diabetes, according to some implementations of the present disclosure; and

FIG. 8 is a flow diagram of a third method for monitoring an individual with diabetes, according to some implementations of the present disclosure.

While the present disclosure is susceptible to various modifications and alternative forms, specific implementations and embodiments thereof have been shown by way of example in the drawings and will herein be described in detail. It should be understood, however, that it is not intended to limit the present disclosure to the particular forms disclosed, but on the contrary, the present disclosure is to cover all modifications, equivalents, and alternatives falling within the spirit and scope of the present disclosure as defined by the appended claims.

### DETAILED DESCRIPTION

The present disclosure is described with reference to the attached figures, where like reference numerals are used throughout the figures to designate similar or equivalent elements. The figures are not drawn to scale, and are provided merely to illustrate the instant disclosure. Several aspects of the disclosure are described below with reference to example applications for illustration.

Many individuals suffer from sleep-related and/or respiratory disorders, such as Periodic Limb Movement Disorder (PLMD), Restless Leg Syndrome (RLS), Sleep-Disordered Breathing (SDB) such as Obstructive Sleep Apnea (OSA), Central Sleep Apnea (CSA) and other types of apneas, Respiratory Effort Related Arousal (RERA), snoring, Cheyne-Stokes Respiration (CSR), respiratory insufficiency, Obesity Hyperventilation Syndrome (OHS), Chronic Obstructive Pulmonary Disease (COPD), Neuromuscular Disease (NMD), and chest wall disorders.

Obstructive Sleep Apnea (OSA), a form of Sleep Disordered Breathing (SDB), is characterized by events including occlusion or obstruction of the upper air passage during sleep resulting from a combination of an abnormally small upper airway and the normal loss of muscle tone in the region of the tongue, soft palate and posterior oropharyngeal wall. Central Sleep Apnea (CSA) is another form of sleep disordered breathing. CSA results when the brain temporarily stops sending signals to the muscles that control breathing. More generally, an apnea generally refers to the cessation of breathing caused by blockage of the air.

Other types of apneas include hypopnea, hyperpnea, and hypercapnia. Hypopnea is generally characterized by slow or shallow breathing caused by a narrowed airway, as opposed to a blocked airway. Hyperpnea is generally characterized by an increase depth and/or rate of breathing. Hypercapnia is generally characterized by elevated or excessive carbon dioxide in the bloodstream, typically caused by inadequate respiration.

A Respiratory Effort Related Arousal (RERA) event is typically characterized by an increased respiratory effort for ten seconds or longer leading to arousal from sleep and which does not fulfill the criteria for an apnea or hypopnea event. RERAs are defined as a sequence of breaths characterized by increasing respiratory effort leading to an arousal from sleep, but which does not meet criteria for an apnea or hypopnea. These events fulfill the following criteria: (1) a pattern of progressively more negative esophageal pressure, terminated by a sudden change in pressure to a less negative level and an arousal, and (2) the event lasts ten seconds or longer. In some implementations, a Nasal Cannula/Pressure Transducer System is adequate and reliable in the detection of RERAs. A RERA detector may be based on a real flow signal derived from a respiratory therapy device. For example, a flow limitation measure may be determined based on a flow signal. A measure of arousal may then be derived as a function of the flow limitation measure and a measure of sudden increase in ventilation. One such method is described in WO 2008/138040 and U.S. Pat. No. 9,358,353, assigned to ResMed Ltd., the disclosure of each of which is hereby incorporated by reference herein in their entireties.

Cheyne-Stokes Respiration (CSR) is another form of sleep-disordered breathing. CSR is a disorder of a patient's respiratory controller in which there are rhythmic alternating periods of waxing and waning ventilation known as CSR cycles. CSR is characterized by repetitive de-oxygenation and re-oxygenation of the arterial blood.

Obesity Hyperventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common, such as increased resistance to air movement, extended expiratory phase of respiration, and loss of the normal elasticity of the lung. COPD encompasses a group of lower airway diseases that have certain characteristics in common, such as increased resistance to air movement, extended expiratory phase of respiration, and loss of the normal elasticity of the lung.

Neuromuscular Disease (NMD) encompasses many diseases and ailments that impair the functioning of the muscles either directly via intrinsic muscle pathology, or indirectly via nerve pathology. Chest wall disorders are a group of thoracic deformities that result in inefficient coupling between the respiratory muscles and the thoracic cage.

These and other disorders are characterized by particular events (e.g., snoring, an apnea, a hypopnea, a restless leg, a sleeping disorder, choking, an increased heart rate, labored breathing, an asthma attack, an epileptic episode, a seizure, or any combination thereof) that can occur when the individual is sleeping.

Individuals with diabetes who also use a respiratory therapy system (for example to treat SDB) can experience positive and/or negative interactions. For example, the use of the respiratory therapy system can impact the efficacy of the individual's diabetes treatment plan (which could include a diabetes medication plan, a diet plan, an exercise plan, etc.). The impact on the efficacy of the individual's diabetes treatment plan can be positive or negative, and thus it can be difficult for these individuals to use a respiratory therapy system in adherence with a respiratory therapy plan, while also adhering to a diabetes treatment plan that remains effective. Thus, it is advantageous to monitor these individuals, and to make various adjustments (and/or determine various adjustments) to their diabetes treatment plans and their use of respiratory therapy systems in order to mitigate, optimize, etc. any interactions between their diabetes treatment plan and their respiratory therapy plan

The Apnea-Hypopnea Index (AHI) is an index used to indicate the severity of sleep apnea during a sleep session. The AHI is calculated by dividing the number of apnea and/or hypopnea events experienced by the user during the sleep session by the total number of hours of sleep in the sleep session. The event can be, for example, a pause in breathing that lasts for at least 10 seconds. An AHI that is less than 5 is considered normal. An AHI that is greater than or equal to 5, but less than 15 is considered indicative of mild sleep apnea. An AHI that is greater than or equal to 15, but less than 30 is considered indicative of moderate sleep apnea. An AHI that is greater than or equal to 30 is considered indicative of severe sleep apnea. In children, an AHI that is greater than 1 is considered abnormal. Sleep apnea can be considered “controlled” when the AHI is normal, or when the AHI is normal or mild. The AHI can also be used in combination with oxygen desaturation levels to indicate the severity of Obstructive Sleep Apnea.

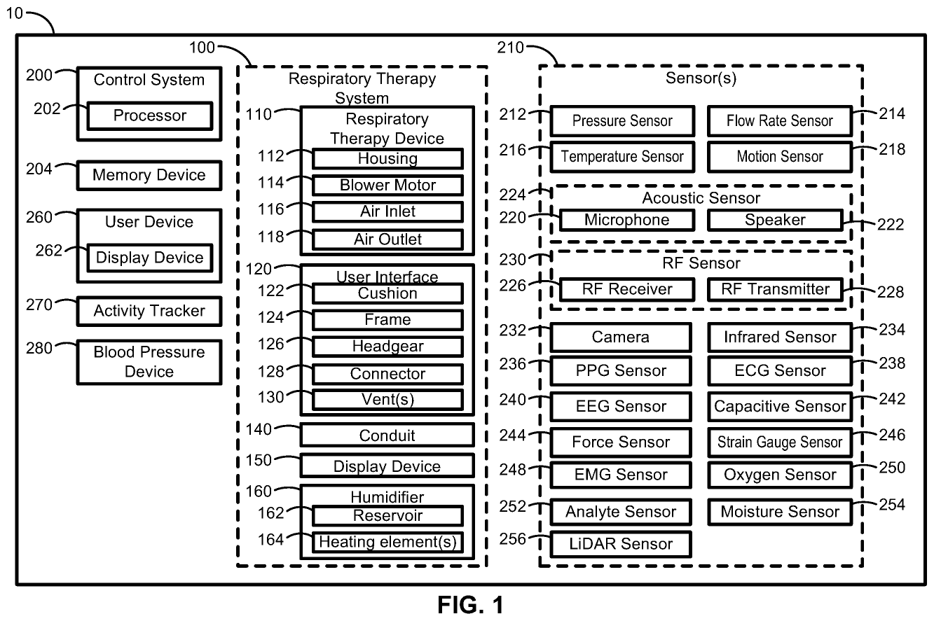

Referring to FIG. 1, a system 10, according to some implementations of the present disclosure, is illustrated. The system 10 can include a respiratory therapy system 100, a control system 200, a memory device 204, and one or more sensors 210. The system 10 may additionally or alternatively include a user device 260, an activity tracker 270, and a blood pressure device 280. The system 10 can be used to monitor an individual with diabetes who uses a respiratory therapy system.

The respiratory therapy system 100 includes a respiratory pressure therapy (RPT) device 110 (referred to herein as respiratory therapy device 110), a user interface 120 (also referred to as a mask or a patient interface), a conduit 140 (also referred to as a tube or an air circuit), a display device 150, and a humidifier 160. Respiratory pressure therapy refers to the application of a supply of air to an entrance to a user's airways at a controlled target pressure that is nominally positive with respect to atmosphere throughout the user's breathing cycle (e.g., in contrast to negative pressure therapies such as the tank ventilator or cuirass). The respiratory therapy system 100 is generally used to treat individuals suffering from one or more sleep-related respiratory disorders (e.g., obstructive sleep apnea, central sleep apnea, or mixed sleep apnea).

The respiratory therapy system 100 can be used, for example, as a ventilator or as a positive airway pressure (PAP) system, such as a continuous positive airway pressure (CPAP) system, an automatic positive airway pressure (APAP) system, a bi-level or variable positive airway pressure (BPAP or VPAP) system, or any combination thereof. The CPAP system delivers a predetermined air pressure (e.g., determined by a sleep physician) to the user. The APAP system automatically varies the air pressure delivered to the user based on, for example, respiration data associated with the user. The BPAP or VPAP system is configured to deliver a first predetermined pressure (e.g., an inspiratory positive airway pressure or IPAP) and a second predetermined pressure (e.g., an expiratory positive airway pressure or EPAP) that is lower than the first predetermined pressure.

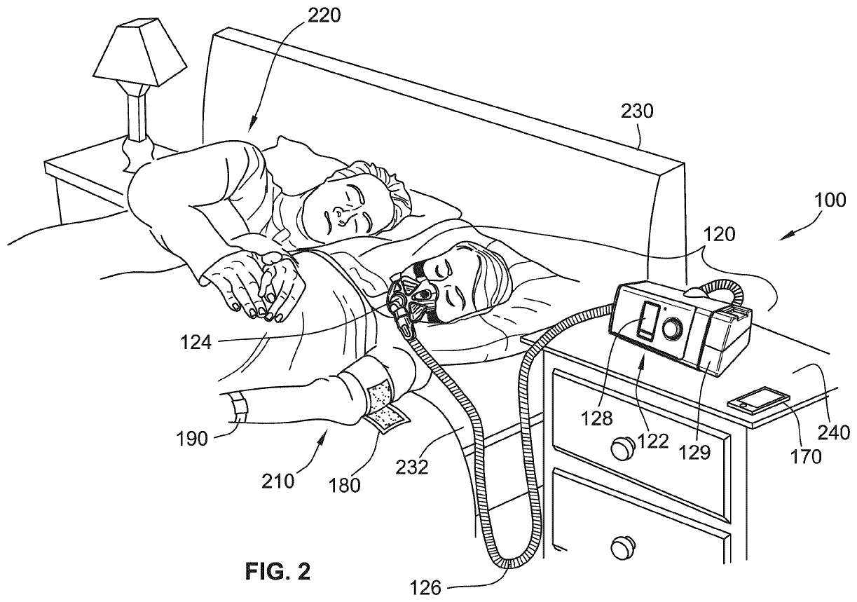

As shown in FIG. 2, the respiratory therapy system 100 can be used to treat a user 20. In this example, the user 20 of the respiratory therapy system 100 and a bed partner 30 are located in a bed 40 and are laying on a mattress 42. The user interface 120 can be worn by the user 20 during a sleep session. The respiratory therapy system 100 generally aids in increasing the air pressure in the throat of the user 20 to aid in preventing the airway from closing and/or narrowing during sleep. The respiratory therapy device 110 can be positioned on a nightstand 44 that is directly adjacent to the bed 40 as shown in FIG. 2, or more generally, on any surface or structure that is generally adjacent to the bed 40 and/or the user 20.

Referring back to FIG. 1, the respiratory therapy device 110 is generally used to generate pressurized air that is delivered to a user (e.g., using one or more motors that drive one or more compressors). In some implementations, the respiratory therapy device 110 generates continuous constant air pressure that is delivered to the user. In other implementations, the respiratory therapy device 110 generates two or more predetermined pressures (e.g., a first predetermined air pressure and a second predetermined air pressure). In still other implementations, the respiratory therapy device 110 generates a variety of different air pressures within a predetermined range. For example, the respiratory therapy device 110 can deliver at least about 6 cmH₂O, at least about 10 cmH₂O, at least about 20 cmH₂O, between about 6 cmH₂O and about 10 cmH₂O, between about 7 cmH₂O and about 12 cmH₂O, etc. The respiratory therapy device 110 can also deliver pressurized air at a predetermined flow rate between, for example, about −20 L/min and about 150 L/min, while maintaining a positive pressure (relative to the ambient pressure).

The respiratory therapy device 110 includes a housing 112, a blower motor 114, an air inlet 116, and an air outlet 118. The blower motor 114 is at least partially disposed or integrated within the housing 112. The blower motor 114 draws air from outside the housing 112 (e.g., atmosphere) via the air inlet 116 and causes pressurized air to flow through the humidifier 160, and through the air outlet 118. In some implementations, the air inlet 116 and/or the air outlet 118 include a cover that is moveable between a closed position and an open position (e.g., to prevent or inhibit air from flowing through the air inlet 116 or the air outlet 118). The housing 112 can also include a vent to allow air to pass through the housing 112 to the air inlet 116. As described below, the conduit 140 is coupled to the air outlet 118 of the respiratory therapy device 110.

The user interface 120 engages a portion of the user's face and delivers pressurized air from the respiratory therapy device 110 to the user's airway to aid in preventing the airway from narrowing and/or collapsing during sleep. This may also increase the user's oxygen intake during sleep. Generally, the user interface 120 engages the user's face such that the pressurized air is delivered to the user's airway via the user's mouth, the user's nose, or both the user's mouth and nose. Together, the respiratory therapy device 110, the user interface 120, and the conduit 140 form an air pathway fluidly coupled with an airway of the user. The pressurized air also increases the user's oxygen intake during sleep. Depending upon the therapy to be applied, the user interface 120 may form a seal, for example, with a region or portion of the user's face, to facilitate the delivery of gas at a pressure at sufficient variance with ambient pressure to effect therapy, for example, at a positive pressure of about 10 cm H₂O relative to ambient pressure. For other forms of therapy, such as the delivery of oxygen, the user interface may not include a seal sufficient to facilitate delivery to the airways of a supply of gas at a positive pressure of about 10 cmH₂O.

The user interface 120 can include, for example, a cushion 122, a frame 124, a headgear 126, connector 128, and one or more vents 130. The cushion 122 and the frame 124 define a volume of space around the mouth and/or nose of the user. When the respiratory therapy system 100 is in use, this volume space receives pressurized air (e.g., from the respiratory therapy device 110 via the conduit 140) for passage into the airway(s) of the user. The headgear 126 is generally used to aid in positioning and/or stabilizing the user interface 120 on a portion of the user (e.g., the face), and along with the cushion 122 (which, for example, can comprise silicone, plastic, foam, etc.) aids in providing a substantially air-tight seal between the user interface 120 and the user 20. In some implementations the headgear 126 includes one or more straps (e.g., including hook and loop fasteners). The connector 128 is generally used to couple (e.g., connect and fluidly couple) the conduit 140 to the cushion 122 and/or frame 124. Alternatively, the conduit 140 can be directly coupled to the cushion 122 and/or frame 124 without the connector 128. The vent 130 can be used for permitting the escape of carbon dioxide and other gases exhaled by the user 20. The user interface 120 generally can include any suitable number of vents (e.g., one, two, five, ten, etc.).

As shown in FIG. 2, in some implementations, the user interface 120 is a facial mask (e.g., a full face mask) that covers at least a portion of the nose and mouth of the user 20. Alternatively, the user interface 120 can be a nasal mask that provides air to the nose of the user or a nasal pillow mask that delivers air directly to the nostrils of the user 20. In other implementations, the user interface 120 includes a mouthpiece (e.g., a night guard mouthpiece molded to conform to the teeth of the user, a mandibular repositioning device, etc.).

Referring back to FIG. 1, the conduit 140 (also referred to as an air circuit or tube) allows the flow of air between components of the respiratory therapy system 100, such as between the respiratory therapy device 110 and the user interface 120. In some implementations, there can be separate limbs of the conduit for inhalation and exhalation. In other implementations, a single limb conduit is used for both inhalation and exhalation.

The conduit 140 includes a first end that is coupled to the air outlet 118 of the respiratory therapy device 110. The first end can be coupled to the air outlet 118 of the respiratory therapy device 110 using a variety of techniques (e.g., a press fit connection, a snap fit connection, a threaded connection, etc.). In some implementations, the conduit 140 includes one or more heating elements that heat the pressurized air flowing through the conduit 140 (e.g., heat the air to a predetermined temperature or within a range of predetermined temperatures). Such heating elements can be coupled to and/or imbedded in the conduit 140. In such implementations, the first end can include an electrical contact that is electrically coupled to the respiratory therapy device 110 to power the one or more heating elements of the conduit 140. For example, the electrical contact can be electrically coupled to an electrical contact of the air outlet 118 of the respiratory therapy device 110. In this example, electrical contact of the conduit 140 can be a male connector and the electrical contact of the air outlet 118 can be female connector, or, alternatively, the opposite configuration can be used.

The display device 150 is generally used to display image(s) including still images, video images, or both and/or information regarding the respiratory therapy device 110. For example, the display device 150 can provide information regarding the status of the respiratory therapy device 110 (e.g., whether the respiratory therapy device 110 is on/off, the pressure of the air being delivered by the respiratory therapy device 110, the temperature of the air being delivered by the respiratory therapy device 110, etc.) and/or other information (e.g., a sleep score and/or a therapy score, also referred to as a my Air™ score, such as described in WO 2016/061629 and U.S. Patent Pub. No. 2017/0311879, which are hereby incorporated by reference herein in their entireties, the current date/time, personal information for the user 20, etc.). In some implementations, the display device 150 acts as a human-machine interface (HMI) that includes a graphic user interface (GUI) configured to display the image(s) as an input interface. The display device 150 can be an LED display, an OLED display, an LCD display, or the like. The input interface can be, for example, a touchscreen or touch-sensitive substrate, a mouse, a keyboard, or any sensor system configured to sense inputs made by a human user interacting with the respiratory therapy device 110.

The humidifier 160 is coupled to or integrated in the respiratory therapy device 110 and includes a reservoir 162 for storing water that can be used to humidify the pressurized air delivered from the respiratory therapy device 110. The humidifier 160 includes a one or more heating elements 164 to heat the water in the reservoir to generate water vapor. The humidifier 160 can be fluidly coupled to a water vapor inlet of the air pathway between the blower motor 114 and the air outlet 118, or can be formed in-line with the air pathway between the blower motor 114 and the air outlet 118. For example, air flows from the air inlet 116 through the blower motor 114, and then through the humidifier 160 before exiting the respiratory therapy device 110 via the air outlet 118

While the respiratory therapy system 100 has been described herein as including each of the respiratory therapy device 110, the user interface 120, the conduit 140, the display device 150, and the humidifier 160, more or fewer components can be included in a respiratory therapy system according to implementations of the present disclosure. For example, a first alternative respiratory therapy system includes the respiratory therapy device 110, the user interface 120, and the conduit 140. As another example, a second alternative system includes the respiratory therapy device 110, the user interface 120, and the conduit 140, and the display device 150. Thus, various respiratory therapy systems can be formed using any portion or portions of the components shown and described herein and/or in combination with one or more other components.

The control system 200 includes one or more processors 202 (hereinafter, processor 202). The control system 200 is generally used to control (e.g., actuate) the various components of the system 10 and/or analyze data obtained and/or generated by the components of the system 10. The processor 202 can be a general or special purpose processor or microprocessor. While one processor 202 is illustrated in FIG. 1, the control system 200 can include any number of processors (e.g., one processor, two processors, five processors, ten processors, etc.) that can be in a single housing, or located remotely from each other. The control system 200 (or any other control system) or a portion of the control system 200 such as the processor 202 (or any other processor(s) or portion(s) of any other control system), can be used to carry out one or more steps of any of the methods described and/or claimed herein. The control system 200 can be coupled to and/or positioned within, for example, a housing of the user device 260, a portion (e.g., the respiratory therapy device 110) of the respiratory therapy system 100, and/or within a housing of one or more of the sensors 210. The control system 200 can be centralized (within one such housing) or decentralized (within two or more of such housings, which are physically distinct). In such implementations including two or more housings containing the control system 200, the housings can be located proximately and/or remotely from each other.

The memory device 204 stores machine-readable instructions that are executable by the processor 202 of the control system 200. The memory device 204 can be any suitable computer readable storage device or media, such as, for example, a random or serial access memory device, a hard drive, a solid state drive, a flash memory device, etc. While one memory device 204 is shown in FIG. 1, the system 10 can include any suitable number of memory devices 204 (e.g., one memory device, two memory devices, five memory devices, ten memory devices, etc.). The memory device 204 can be coupled to and/or positioned within a housing of a respiratory therapy device 110 of the respiratory therapy system 100, within a housing of the user device 260, within a housing of one or more of the sensors 210, or any combination thereof. Like the control system 200, the memory device 204 can be centralized (within one such housing) or decentralized (within two or more of such housings, which are physically distinct).

In some implementations, the memory device 204 stores a user profile associated with the user. The user profile can include, for example, demographic information associated with the user, biometric information associated with the user, medical information associated with the user, self-reported user feedback, sleep parameters associated with the user (e.g., sleep-related parameters recorded from one or more earlier sleep sessions), or any combination thereof. The demographic information can include, for example, information indicative of an age of the user, a gender of the user, a race of the user, a geographic location of the user, a relationship status, a family history of insomnia or sleep apnea, an employment status of the user, an educational status of the user, a socioeconomic status of the user, or any combination thereof. The medical information can include, for example, information indicative of one or more medical conditions associated with the user, medication usage by the user, or both. The medical information data can further include a multiple sleep latency test (MSLT) result or score and/or a Pittsburgh Sleep Quality Index (PSQI) score or value. The self-reported user feedback can include information indicative of a self-reported subjective sleep score (e.g., poor, average, excellent), a self-reported subjective stress level of the user, a self-reported subjective fatigue level of the user, a self-reported subjective health status of the user, a recent life event experienced by the user, or any combination thereof.

As described herein, the processor 202 and/or memory device 204 can receive data (e.g., physiological data and/or audio data) from the one or more sensors 210 such that the data for storage in the memory device 204 and/or for analysis by the processor 202. The processor 202 and/or memory device 204 can communicate with the one or more sensors 210 using a wired connection or a wireless connection (e.g., using an RF communication protocol, a Wi-Fi communication protocol, a Bluetooth communication protocol, over a cellular network, etc.). In some implementations, the system 10 can include an antenna, a receiver (e.g., an RF receiver), a transmitter (e.g., an RF transmitter), a transceiver, or any combination thereof. Such components can be coupled to or integrated a housing of the control system 200 (e.g., in the same housing as the processor 202 and/or memory device 204), or the user device 260.

The one or more sensors 210 include a pressure sensor 212, a flow rate sensor 214, temperature sensor 216, a motion sensor 218, a microphone 220, a speaker 222, a radio-frequency (RF) receiver 226, a RF transmitter 228, a camera 232, an infrared (IR) sensor 234, a photoplethysmogram (PPG) sensor 236, an electrocardiogram (ECG) sensor 238, an electroencephalography (EEG) sensor 240, a capacitive sensor 242, a force sensor 244, a strain gauge sensor 246, an electromyography (EMG) sensor 248, an oxygen sensor 250, an analyte sensor 252, a moisture sensor 254, a Light Detection and Ranging (LiDAR) sensor 256, or any combination thereof. Generally, each of the one or more sensors 210 are configured to output sensor data that is received and stored in the memory device 204 or one or more other memory devices.

While the one or more sensors 210 are shown and described as including each of the pressure sensor 212, the flow rate sensor 214, the temperature sensor 216, the motion sensor 218, the microphone 220, the speaker 222, the RF receiver 226, the RF transmitter 228, the camera 232, the IR sensor 234, the PPG sensor 236, the ECG sensor 238, the EEG sensor 240, the capacitive sensor 242, the force sensor 244, the strain gauge sensor 246, the EMG sensor 248, the oxygen sensor 250, the analyte sensor 252, the moisture sensor 254, and the LiDAR sensor 256, more generally, the one or more sensors 210 can include any combination and any number of each of the sensors described and/or shown herein.

As described herein, the system 10 generally can be used to generate physiological data associated with a user (e.g., a user of the respiratory therapy system 100) during a sleep session. The physiological data can be analyzed to generate one or more sleep-related parameters, which can include any parameter, measurement, etc. related to the user during the sleep session. The one or more sleep-related parameters that can be determined for the user 20 during the sleep session include, for example, an Apnea-Hypopnea Index (AHI) score, a sleep score, a flow signal, a respiration signal, a respiration rate, an inspiration amplitude, an expiration amplitude, an inspiration-expiration ratio, a number of events per hour, a pattern of events, a stage, pressure settings of the respiratory therapy device 110, a heart rate, a heart rate variability, movement of the user 20, temperature, EEG activity, EMG activity, arousal, snoring, choking, coughing, whistling, wheezing, or any combination thereof.

The one or more sensors 210 can be used to generate, for example, physiological data, audio data, or both. Physiological data generated by one or more of the sensors 210 can be used by the control system 200 to determine a sleep-wake signal associated with the user 20 during the sleep session and one or more sleep-related parameters. The sleep-wake signal can be indicative of one or more sleep states, including wakefulness, relaxed wakefulness, micro-awakenings, or distinct sleep stages such as, for example, a rapid eye movement (REM) stage, a first non-REM stage (often referred to as “N1”), a second non-REM stage (often referred to as “N2”), a third non-REM stage (often referred to as “N3”), or any combination thereof. Methods for determining sleep states and/or sleep stages from physiological data generated by one or more sensors, such as the one or more sensors 210, are described in, for example, WO 2014/047310, U.S. Patent Pub. No. 2014/0088373, WO 2017/132726, WO 2019/122413, WO 2019/122414, and U.S. Patent Pub. No. 2020/0383580 each of which is hereby incorporated by reference herein in its entirety.

In some implementations, the sleep-wake signal described herein can be timestamped to indicate a time that the user enters the bed, a time that the user exits the bed, a time that the user attempts to fall asleep, etc. The sleep-wake signal can be measured by the one or more sensors 210 during the sleep session at a predetermined sampling rate, such as, for example, one sample per second, one sample per 30 seconds, one sample per minute, etc. In some implementations, the sleep-wake signal can also be indicative of a respiration signal, a respiration rate, an inspiration amplitude, an expiration amplitude, an inspiration-expiration ratio, a number of events per hour, a pattern of events, pressure settings of the respiratory therapy device 110, or any combination thereof during the sleep session. The event(s) can include snoring, apneas, central apneas, obstructive apneas, mixed apneas, hypopneas, a mask leak (e.g., from the user interface 120), a restless leg, a sleeping disorder, choking, an increased heart rate, labored breathing, an asthma attack, an epileptic episode, a seizure, or any combination thereof. The one or more sleep-related parameters that can be determined for the user during the sleep session based on the sleep-wake signal include, for example, a total time in bed, a total sleep time, a sleep onset latency, a wake-after-sleep-onset parameter, a sleep efficiency, a fragmentation index, or any combination thereof. As described in further detail herein, the physiological data and/or the sleep-related parameters can be analyzed to determine one or more sleep-related scores.

Physiological data and/or audio data generated by the one or more sensors 210 can also be used to determine a respiration signal associated with a user during a sleep session. The respiration signal is generally indicative of respiration or breathing of the user during the sleep session. The respiration signal can be indicative of and/or analyzed to determine (e.g., using the control system 200) one or more sleep-related parameters, such as, for example, a respiration rate, a respiration rate variability, an inspiration amplitude, an expiration amplitude, an inspiration-expiration ratio, an occurrence of one or more events, a number of events per hour, a pattern of events, a sleep state, a sleet stage, an apnea-hypopnea index (AHI), pressure settings of the respiratory therapy device 110, or any combination thereof. The one or more events can include snoring, apneas, central apneas, obstructive apneas, mixed apneas, hypopneas, a mask leak (e.g., from the user interface 120), a cough, a restless leg, a sleeping disorder, choking, an increased heart rate, labored breathing, an asthma attack, an epileptic episode, a seizure, increased blood pressure, or any combination thereof. Many of the described sleep-related parameters are physiological parameters, although some of the sleep-related parameters can be considered to be non-physiological parameters. Other types of physiological and/or non-physiological parameters can also be determined, either from the data from the one or more sensors 210, or from other types of data.

The pressure sensor 212 outputs pressure data that can be stored in the memory device 204 and/or analyzed by the processor 202 of the control system 200. In some implementations, the pressure sensor 212 is an air pressure sensor (e.g., barometric pressure sensor) that generates sensor data indicative of the respiration (e.g., inhaling and/or exhaling) of the user of the respiratory therapy system 100 and/or ambient pressure. In such implementations, the pressure sensor 212 can be coupled to or integrated in the respiratory therapy device 110. The pressure sensor 212 can be, for example, a capacitive sensor, an electromagnetic sensor, a piezoelectric sensor, a strain-gauge sensor, an optical sensor, a potentiometric sensor, or any combination thereof.

The flow rate sensor 214 outputs flow rate data that can be stored in the memory device 204 and/or analyzed by the processor 202 of the control system 200. Examples of flow rate sensors (such as, for example, the flow rate sensor 214) are described in International Publication No. WO 2012/012835 and U.S. Pat. No. 10,328,219, both of which are hereby incorporated by reference herein in their entireties. In some implementations, the flow rate sensor 214 is used to determine an air flow rate from the respiratory therapy device 110, an air flow rate through the conduit 140, an air flow rate through the user interface 120, or any combination thereof. In such implementations, the flow rate sensor 214 can be coupled to or integrated in the respiratory therapy device 110, the user interface 120, or the conduit 140. The flow rate sensor 214 can be a mass flow rate sensor such as, for example, a rotary flow meter (e.g., Hall effect flow meters), a turbine flow meter, an orifice flow meter, an ultrasonic flow meter, a hot wire sensor, a vortex sensor, a membrane sensor, or any combination thereof. In some implementations, the flow rate sensor 214 is configured to measure a vent flow (e.g., intentional “leak”), an unintentional leak (e.g., mouth leak and/or mask leak), a patient flow (e.g., air into and/or out of lungs), or any combination thereof. In some implementations, the flow rate data can be analyzed to determine cardiogenic oscillations of the user. In some examples, the pressure sensor 212 can be used to determine a blood pressure of a user.

The temperature sensor 216 outputs temperature data that can be stored in the memory device 204 and/or analyzed by the processor 202 of the control system 200. In some implementations, the temperature sensor 216 generates temperatures data indicative of a core body temperature of the user 20, a skin temperature of the user 20, a temperature of the air flowing from the respiratory therapy device 110 and/or through the conduit 140, a temperature in the user interface 120, an ambient temperature, or any combination thereof. The temperature sensor 216 can be, for example, a thermocouple sensor, a thermistor sensor, a silicon band gap temperature sensor or semiconductor-based sensor, a resistance temperature detector, or any combination thereof.

The motion sensor 218 outputs motion data that can be stored in the memory device 204 and/or analyzed by the processor 202 of the control system 200. The motion sensor 218 can be used to detect movement of the user 20 during the sleep session, and/or detect movement of any of the components of the respiratory therapy system 100, such as the respiratory therapy device 110, the user interface 120, or the conduit 140. The motion sensor 218 can include one or more inertial sensors, such as accelerometers, gyroscopes, and magnetometers. In some implementations, the motion sensor 218 alternatively or additionally generates one or more signals representing bodily movement of the user, from which may be obtained a signal representing a sleep state of the user; for example, via a respiratory movement of the user. In some implementations, the motion data from the motion sensor 218 can be used in conjunction with additional data from another one of the sensors 210 to determine the sleep state of the user.

The microphone 220 outputs sound and/or audio data that can be stored in the memory device 204 and/or analyzed by the processor 202 of the control system 200. The audio data generated by the microphone 220 is reproducible as one or more sound(s) during a sleep session (e.g., sounds from the user 20). The audio data form the microphone 220 can also be used to identify (e.g., using the control system 200) an event experienced by the user during the sleep session, as described in further detail herein. The microphone 220 can be coupled to or integrated in the respiratory therapy device 110, the user interface 120, the conduit 140, or the user device 260. The microphone 220 can be coupled to or integrated in a wearable device, such as a smartwatch, smart glasses, earphones or ear buds, or other head wearable device. In some implementations, the system 10 includes a plurality of microphones (e.g., two or more microphones and/or an array of microphones with beamforming) such that sound data generated by each of the plurality of microphones can be used to discriminate the sound data generated by another of the plurality of microphones.

The speaker 222 outputs sound waves that are audible to a user of the system 10 (e.g., the user 20 of FIG. 2). The speaker 222 can be used, for example, as an alarm clock or to play an alert or message to the user 20 (e.g., in response to an event). In some implementations, the speaker 222 can be used to communicate the audio data generated by the microphone 220 to the user. The speaker 222 can be coupled to or integrated in the respiratory therapy device 110, the user interface 120, the conduit 140, or the user device 260, and/or can be coupled to or integrated in a wearable device, such as a smartwatch, smart glasses, earphones or ear buds, or other head wearable device.

The microphone 220 and the speaker 222 can be used as separate devices. In some implementations, the microphone 220 and the speaker 222 can be combined into an acoustic sensor 224 (e.g., a sonar sensor), as described in, for example, WO 2018/050913, WO 2020/104465, U.S. Pat. App. Pub. No. 2022/0007965, each of which is hereby incorporated by reference herein in its entirety. In such implementations, the speaker 222 generates or emits sound waves at a predetermined interval and the microphone 220 detects the reflections of the emitted sound waves from the speaker 222. The sound waves generated or emitted by the speaker 222 have a frequency that is not audible to the human ear (e.g., below 20 Hz or above around 18 kHz) so as not to disturb the sleep of the user 20 or the bed partner 30. Based at least in part on the data from the microphone 220 and/or the speaker 222, the control system 200 can determine a location of the user 20 and/or one or more of the sleep-related parameters described in herein such as, for example, a respiration signal, a respiration rate, an inspiration amplitude, an expiration amplitude, an inspiration-expiration ratio, a number of events per hour, a pattern of events, a sleep state, a sleep stage, pressure settings of the respiratory therapy device 110, or any combination thereof. In such a context, a sonar sensor may be understood to concern an active acoustic sensing, such as by generating and/or transmitting ultrasound and/or low frequency ultrasound sensing signals (e.g., in a frequency range of about 17-23 kHz, 18-22 kHz, or 17-18 kHz, for example), through the air.

In some implementations, the sensors 210 include (i) a first microphone that is the same as, or similar to, the microphone 220, and is integrated in the acoustic sensor 224 and (ii) a second microphone that is the same as, or similar to, the microphone 220, but is separate and distinct from the first microphone that is integrated in the acoustic sensor 224.

The RF transmitter 228 generates and/or emits radio waves having a predetermined frequency and/or a predetermined amplitude (e.g., within a high frequency band, within a low frequency band, long wave signals, short wave signals, etc.). The RF receiver 226 detects the reflections of the radio waves emitted from the RF transmitter 228, and this data can be analyzed by the control system 200 to determine a location of the user and/or one or more of the sleep-related parameters described herein. An RF receiver (either the RF receiver 226 and the RF transmitter 228 or another RF pair) can also be used for wireless communication between the control system 200, the respiratory therapy device 110, the one or more sensors 210, the user device 260, or any combination thereof. While the RF receiver 226 and RF transmitter 228 are shown as being separate and distinct elements in FIG. 1, in some implementations, the RF receiver 226 and RF transmitter 228 are combined as a part of an RF sensor 230 (e.g., a RADAR sensor). In some such implementations, the RF sensor 230 includes a control circuit. The format of the RF communication can be Wi-Fi, Bluetooth, or the like.

In some implementations, the RF sensor 230 is a part of a mesh system. One example of a mesh system is a Wi-Fi mesh system, which can include mesh nodes, mesh router(s), and mesh gateway(s), each of which can be mobile/movable or fixed. In such implementations, the Wi-Fi mesh system includes a Wi-Fi router and/or a Wi-Fi controller and one or more satellites (e.g., access points), each of which include an RF sensor that the is the same as, or similar to, the RF sensor 230. The Wi-Fi router and satellites continuously communicate with one another using Wi-Fi signals. The Wi-Fi mesh system can be used to generate motion data based on changes in the Wi-Fi signals (e.g., differences in received signal strength) between the router and the satellite(s) due to an object or person moving partially obstructing the signals. The motion data can be indicative of motion, breathing, heart rate, gait, falls, behavior, etc., or any combination thereof.

The camera 232 outputs image data reproducible as one or more images (e.g., still images, video images, thermal images, or any combination thereof) that can be stored in the memory device 204. The image data from the camera 232 can be used by the control system 200 to determine one or more of the sleep-related parameters described herein, such as, for example, one or more events (e.g., periodic limb movement or restless leg syndrome), a respiration signal, a respiration rate, an inspiration amplitude, an expiration amplitude, an inspiration-expiration ratio, a number of events per hour, a pattern of events, a sleep state, a sleep stage, or any combination thereof. Further, the image data from the camera 232 can be used to, for example, identify a location of the user, to determine chest movement of the user, to determine air flow of the mouth and/or nose of the user, to determine a time when the user enters the bed, and to determine a time when the user exits the bed. In some implementations, the camera 232 includes a wide angle lens or a fish eye lens.

The IR sensor 234 outputs infrared image data reproducible as one or more infrared images (e.g., still images, video images, or both) that can be stored in the memory device 204. The infrared data from the IR sensor 234 can be used to determine one or more sleep-related parameters during a sleep session, including a temperature of the user 20 and/or movement of the user 20. The IR sensor 234 can also be used in conjunction with the camera 232 when measuring the presence, location, and/or movement of the user 20. The IR sensor 234 can detect infrared light having a wavelength between about 700 nm and about 1 mm, for example, while the camera 232 can detect visible light having a wavelength between about 380 nm and about 740 nm.

The PPG sensor 236 outputs physiological data associated with the user 20 that can be used to determine one or more sleep-related parameters, such as, for example, a heart rate, a heart rate variability, a cardiac cycle, respiration rate, an inspiration amplitude, an expiration amplitude, an inspiration-expiration ratio, estimated blood pressure parameter(s), or any combination thereof. The PPG sensor 236 can be worn by the user 20, embedded in clothing and/or fabric that is worn by the user 20, embedded in and/or coupled to the user interface 120 and/or its associated headgear (e.g., straps, etc.), etc.

The ECG sensor 238 outputs physiological data associated with electrical activity of the heart of the user 20. In some implementations, the ECG sensor 238 includes one or more electrodes that are positioned on or around a portion of the user 20 during the sleep session. The physiological data from the ECG sensor 238 can be used, for example, to determine one or more of the sleep-related parameters described herein.

The EEG sensor 240 outputs physiological data associated with electrical activity of the brain of the user 20. In some implementations, the EEG sensor 240 includes one or more electrodes that are positioned on or around the scalp of the user 20 during the sleep session. The physiological data from the EEG sensor 240 can be used, for example, to determine a sleep state and/or a sleep stage of the user 20 at any given time during the sleep session. In some implementations, the EEG sensor 240 can be integrated into the user interface 120, into associated headgear (e.g., straps, etc.), into a head band or other head-worn sensor device, etc.

The capacitive sensor 242, the force sensor 244, and the strain gauge sensor 246 output data that can be stored in the memory device 204 and used/analyzed by the control system 200 to determine, for example, one or more of the sleep-related parameters described herein. The EMG sensor 248 outputs physiological data associated with electrical activity produced by one or more muscles. The oxygen sensor 250 outputs oxygen data indicative of an oxygen concentration of gas (e.g., in the conduit 140 or at the user interface 120). The oxygen sensor 250 can be, for example, an ultrasonic oxygen sensor, an electrical oxygen sensor, a chemical oxygen sensor, an optical oxygen sensor, a pulse oximeter (e.g., SpO₂ sensor), or any combination thereof.

The analyte sensor 252 can be used to detect the presence of an analyte in the exhaled breath of the user 20. The data output by the analyte sensor 252 can be stored in the memory device 204 and used by the control system 200 to determine the identity and concentration of any analytes in the breath of the user. In some implementations, the analyte sensor 252 is positioned near a mouth of the user to detect analytes in breath exhaled from the user's mouth. For example, when the user interface 120 is a facial mask that covers the nose and mouth of the user, the analyte sensor 252 can be positioned within the facial mask to monitor the user's mouth breathing. In other implementations, such as when the user interface 120 is a nasal mask or a nasal pillow mask, the analyte sensor 252 can be positioned near the nose of the user to detect analytes in breath exhaled through the user's nose. In still other implementations, the analyte sensor 252 can be positioned near the user's mouth when the user interface 120 is a nasal mask or a nasal pillow mask. In this implementation, the analyte sensor 252 can be used to detect whether any air is inadvertently leaking from the user's mouth and/or the user interface 120. In some implementations, the analyte sensor 252 is a volatile organic compound (VOC) sensor that can be used to detect carbon-based chemicals or compounds. In some implementations, the analyte sensor 252 can also be used to detect whether the user is breathing through their nose or mouth. For example, if the data output by an analyte sensor 252 positioned near the mouth of the user or within the facial mask (e.g., in implementations where the user interface 120 is a facial mask) detects the presence of an analyte, the control system 200 can use this data as an indication that the user is breathing through their mouth.

The moisture sensor 254 outputs data that can be stored in the memory device 204 and used by the control system 200. The moisture sensor 254 can be used to detect moisture in various areas surrounding the user (e.g., inside the conduit 140 or the user interface 120, near the user's face, near the connection between the conduit 140 and the user interface 120, near the connection between the conduit 140 and the respiratory therapy device 110, etc.). Thus, in some implementations, the moisture sensor 254 can be coupled to or integrated in the user interface 120 or in the conduit 140 to monitor the humidity of the pressurized air from the respiratory therapy device 110. In other implementations, the moisture sensor 254 is placed near any area where moisture levels need to be monitored. The moisture sensor 254 can also be used to monitor the humidity of the ambient environment surrounding the user, for example, the air inside the bedroom.

The LiDAR sensor 256 can be used for depth sensing. This type of optical sensor (e.g., laser sensor) can be used to detect objects and build three dimensional (3D) maps of the surroundings, such as of a living space. LiDAR can generally utilize a pulsed laser to make time of flight measurements. LiDAR is also referred to as 3D laser scanning. In an example of use of such a sensor, a fixed or mobile device (such as a smartphone) having a LiDAR sensor 256 can measure and map an area extending 5 meters or more away from the sensor. The LiDAR data can be fused with point cloud data estimated by an electromagnetic RADAR sensor, for example. The LiDAR sensor(s) 256 can also use artificial intelligence (AI) to automatically geofence RADAR systems by detecting and classifying features in a space that might cause issues for RADAR systems, such a glass windows (which can be highly reflective to RADAR). LiDAR can also be used to provide an estimate of the height of a person, as well as changes in height when the person sits down, or falls down, for example. LiDAR may be used to form a 3D mesh representation of an environment. In a further use, for solid surfaces through which radio waves pass (e.g., radio-translucent materials), the LiDAR may reflect off such surfaces, thus allowing a classification of different type of obstacles.

In some implementations, the one or more sensors 210 also include a galvanic skin response (GSR) sensor, a blood flow sensor, a respiration sensor, a pulse sensor, a sphygmomanometer sensor, an oximetry sensor, a sonar sensor, a RADAR sensor, a blood glucose sensor, a color sensor, a pH sensor, an air quality sensor, a tilt sensor, a rain sensor, a soil moisture sensor, a water flow sensor, an alcohol sensor, or any combination thereof.

While shown separately in FIG. 1, any combination of the one or more sensors 210 can be integrated in and/or coupled to any one or more of the components of the system 10, including the respiratory therapy device 110, the user interface 120, the conduit 140, the humidifier 160, the control system 200, the user device 260, the activity tracker 270, or any combination thereof. For example, the microphone 220 and the speaker 222 can be integrated in and/or coupled to the user device 260 and the pressure sensor 212 and/or flow rate sensor 214 are integrated in and/or coupled to the respiratory therapy device 110. In some implementations, at least one of the one or more sensors 210 is not coupled to the respiratory therapy device 110, the control system 200, or the user device 260, and is positioned generally adjacent to the user 20 during the sleep session (e.g., positioned on or in contact with a portion of the user 20, worn by the user 20, coupled to or positioned on the nightstand, coupled to the mattress, coupled to the ceiling, etc.).

One or more of the respiratory therapy device 110, the user interface 120, the conduit 140, the display device 150, and the humidifier 160 can contain one or more sensors (e.g., a pressure sensor, a flow rate sensor, or more generally any of the other sensors 210 described herein). These one or more sensors can be used, for example, to measure the air pressure and/or flow rate of pressurized air supplied by the respiratory therapy device 110.

The data from the one or more sensors 210 can be analyzed (e.g., by the control system 200) to determine one or more sleep-related parameters, which can include a respiration signal, a respiration rate, a respiration pattern, an inspiration amplitude, an expiration amplitude, an inspiration-expiration ratio, an occurrence of one or more events, a number of events per hour, a pattern of events, a sleep state, an apnea-hypopnea index (AHI), or any combination thereof. The one or more events can include snoring, apneas, central apneas, obstructive apneas, mixed apneas, hypopneas, a mask leak, a cough, a restless leg, a sleeping disorder, choking, an increased heart rate, labored breathing, an asthma attack, an epileptic episode, a seizure, increased blood pressure, or any combination thereof. Many of these sleep-related parameters are physiological parameters, although some of the sleep-related parameters can be considered to be non-physiological parameters. Other types of physiological and non-physiological parameters can also be determined, either from the data from the one or more sensors 210, or from other types of data.

The user device 260 includes a display device 262. The user device 260 can be, for example, a mobile device such as a smart phone, a tablet, a gaming console, a smart watch, a laptop, or the like. Alternatively, the user device 260 can be an external sensing system, a television (e.g., a smart television) or another smart home device (e.g., a smart speaker(s) such as Google Home™, Google Nest™ Amazon Echo™, Amazon Echo Show™, Alexa™-enabled devices, etc.). In some implementations, the user device is a wearable device (e.g., a smart watch). The display device 262 is generally used to display image(s) including still images, video images, or both. In some implementations, the display device 262 acts as a human-machine interface (HMI) that includes a graphic user interface (GUI) configured to display the image(s) and an input interface. The display device 262 can be an LED display, an OLED display, an LCD display, or the like. The input interface can be, for example, a touchscreen or touch-sensitive substrate, a mouse, a keyboard, or any sensor system configured to sense inputs made by a human user interacting with the user device 260. In some implementations, one or more user devices can be used by and/or included in the system 10.

In some implementations, the system 10 also includes the activity tracker 270. The activity tracker 270 is generally used to aid in generating physiological data associated with the user. The activity tracker 270 can include one or more of the sensors 210 described herein, such as, for example, the motion sensor 218 (e.g., one or more accelerometers and/or gyroscopes), the PPG sensor 236, and/or the ECG sensor 238. The physiological data from the activity tracker 270 can be used to determine, for example, a number of steps, a distance traveled, a number of steps climbed, a duration of physical activity, a type of physical activity, an intensity of physical activity, time spent standing, a respiration rate, an average respiration rate, a resting respiration rate, a maximum he respiration art rate, a respiration rate variability, a heart rate, an average heart rate, a resting heart rate, a maximum heart rate, a heart rate variability, a number of calories burned, blood oxygen saturation, electrodermal activity (also known as skin conductance or galvanic skin response), or any combination thereof. In some implementations, the activity tracker 270 is coupled (e.g., electronically or physically) to the user device 260.

In some implementations, the activity tracker 270 is a wearable device that can be worn by the user, such as a smartwatch, a wristband, a ring, or a patch. For example, referring to FIG. 2, the activity tracker 270 is worn on a wrist of the user 20. The activity tracker 270 can also be coupled to or integrated a garment or clothing that is worn by the user. Alternatively still, the activity tracker 270 can also be coupled to or integrated in (e.g., within the same housing) the user device 260. More generally, the activity tracker 270 can be communicatively coupled with, or physically integrated in (e.g., within a housing), the control system 200, the memory device 204, the respiratory therapy system 100, and/or the user device 260.

In some implementations, the system 10 also includes the blood pressure device 280. The blood pressure device 280 is generally used to aid in generating cardiovascular data for determining one or more blood pressure measurements associated with the user 20. The blood pressure device 280 can include at least one of the one or more sensors 210 to measure, for example, a systolic blood pressure component and/or a diastolic blood pressure component.

In some implementations, the blood pressure device 280 is a sphygmomanometer including an inflatable cuff that can be worn by the user 20 and a pressure sensor (e.g., the pressure sensor 212 described herein). For example, in the example of FIG. 2, the blood pressure device 280 can be worn on an upper arm of the user 20. In such implementations where the blood pressure device 280 is a sphygmomanometer, the blood pressure device 280 also includes a pump (e.g., a manually operated bulb) for inflating the cuff. In some implementations, the blood pressure device 280 is coupled to the respiratory therapy device 110 of the respiratory therapy system 100, which in turn delivers pressurized air to inflate the cuff. More generally, the blood pressure device 280 can be communicatively coupled with, and/or physically integrated in (e.g., within a housing), the control system 200, the memory device 204, the respiratory therapy system 100, the user device 260, and/or the activity tracker 270.

In other implementations, the blood pressure device 280 is an ambulatory blood pressure monitor communicatively coupled to the respiratory therapy system 100. An ambulatory blood pressure monitor includes a portable recording device attached to a belt or strap worn by the user 20 and an inflatable cuff attached to the portable recording device and worn around an arm of the user 20. The ambulatory blood pressure monitor is configured to measure blood pressure between about every fifteen minutes to about thirty minutes over a 24-hour or a 48-hour period. The ambulatory blood pressure monitor may measure heart rate of the user 20 at the same time. These multiple readings are averaged over the 24-hour period. The ambulatory blood pressure monitor determines any changes in the measured blood pressure and heart rate of the user 20, as well as any distribution and/or trending patterns of the blood pressure and heart rate data during a sleeping period and an awakened period of the user 20. The measured data and statistics may then be communicated to the respiratory therapy system 100.

The blood pressure device 280 maybe positioned external to the respiratory therapy system 100, coupled directly or indirectly to the user interface 120, coupled directly or indirectly to headgear 126 (if present with the version of the user interface 120 being used), or inflatably coupled to or about a portion of the user 20. The blood pressure device 280 is generally used to aid in generating physiological data for determining one or more blood pressure measurements associated with a user, for example, a systolic blood pressure component and/or a diastolic blood pressure component. In some implementations, the blood pressure device 280 is a sphygmomanometer including an inflatable cuff that can be worn by a user and a pressure sensor (e.g., the pressure sensor 212 described herein).

In some implementations, the blood pressure device 280 is an invasive device which can continuously monitor arterial blood pressure of the user 20 and take an arterial blood sample on demand for analyzing gas of the arterial blood. In some other implementations, the blood pressure device 280 is a continuous blood pressure monitor, using a radio frequency sensor and capable of measuring blood pressure of the user 20 once very few seconds (e.g., every 3 seconds, every 5 seconds, every 7 seconds, etc.) The radio frequency sensor may use continuous wave, frequency-modulated continuous wave (FMCW with ramp chirp, triangle, sinewave), other schemes such as phase-shift keying (PSK), frequency-shift keying (FSK) etc., pulsed continuous wave, and/or spread in ultra wideband ranges (which may include spreading, pseudorandom noise (PRN) codes, or impulse systems).

While the control system 200 and the memory device 204 are described and shown in FIG. 1 as being a separate and distinct component of the system 10, in some implementations, the control system 200 and/or the memory device 204 are integrated in the user device 260 and/or the respiratory therapy device 110. Alternatively, in some implementations, the control system 200 or a portion thereof (e.g., the processor 202) can be located in a cloud (e.g., integrated in a server, integrated in an Internet of Things (IoT) device, connected to the cloud, be subject to edge cloud processing, etc.), located in one or more servers (e.g., remote servers, local servers, etc., or any combination thereof.

While system 10 is shown as including all of the components described above, more or

fewer components can be included in a system according to implementations of the present disclosure. For example, a first alternative system includes the control system 200, the memory device 204, and at least one of the one or more sensors 210 and does not include the respiratory therapy system 100. As another example, a second alternative system includes the control system 200, the memory device 204, at least one of the one or more sensors 210, and the user device 260. As yet another example, a third alternative system includes the control system 200, the memory device 204, the respiratory therapy system 100, at least one of the one or more sensors 210, and the user device 260. Thus, various systems can be formed using any portion or portions of the components shown and described herein and/or in combination with one or more other components.

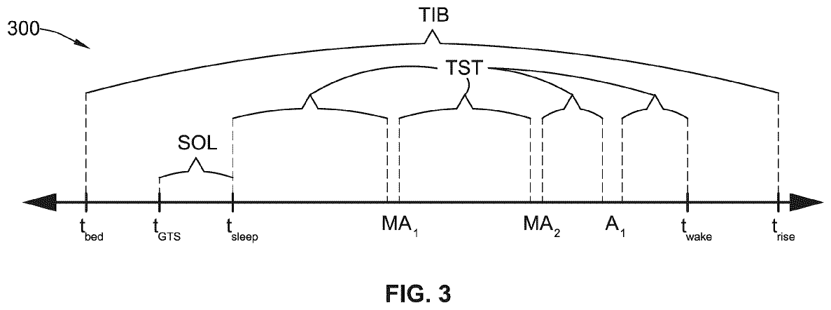

Referring now to FIG. 3, as used herein, a sleep session can be defined multiple ways. For example, a sleep session can be defined by an initial start time and an end time. In some implementations, a sleep session is a duration where the user is asleep, that is, the sleep session has a start time and an end time, and during the sleep session, the user does not wake until the end time. That is, any period of the user being awake is not included in a sleep session. From this first definition of sleep session, if the user wakes ups and falls asleep multiple times in the same night, each of the sleep intervals separated by an awake interval is a sleep session.

Alternatively, in some implementations, a sleep session has a start time and an end time, and during the sleep session, the user can wake up, without the sleep session ending, so long as a continuous duration that the user is awake is below an awake duration threshold. The awake duration threshold can be defined as a percentage of a sleep session. The awake duration threshold can be, for example, about twenty percent of the sleep session, about fifteen percent of the sleep session duration, about ten percent of the sleep session duration, about five percent of the sleep session duration, about two percent of the sleep session duration, etc., or any other threshold percentage. In some implementations, the awake duration threshold is defined as a fixed amount of time, such as, for example, about one hour, about thirty minutes, about fifteen minutes, about ten minutes, about five minutes, about two minutes, etc., or any other amount of time.

In some implementations, a sleep session is defined as the entire time between the time in the evening at which the user first entered the bed, and the time the next morning when user last left the bed. Put another way, a sleep session can be defined as a period of time that begins on a first date (e.g., Monday, Jan. 6, 2020) at a first time (e.g., 10:00 PM), that can be referred to as the current evening, when the user first enters a bed with the intention of going to sleep (e.g., not if the user intends to first watch television or play with a smart phone before going to sleep, etc.), and ends on a second date (e.g., Tuesday, Jan. 7, 2020) at a second time (e.g., 7:00 AM), that can be referred to as the next morning, when the user first exits the bed with the intention of not going back to sleep that next morning.

In some implementations, the user can manually define the beginning of a sleep session and/or manually terminate a sleep session. For example, the user can select (e.g., by clicking or tapping) one or more user-selectable element that is displayed on the display device 262 of the user device 260 (FIG. 1) to manually initiate or terminate the sleep session.

Generally, the sleep session includes any point in time after the user has laid or sat down in the bed (or another area or object on which they intend to sleep), and has turned on the respiratory therapy device 110 and donned the user interface 120. The sleep session can thus include time periods (i) when the user is using the respiratory therapy system 100, but before the user attempts to fall asleep (for example when the user lays in the bed reading a book); (ii) when the user begins trying to fall asleep but is still awake; (iii) when the user is in a light sleep (also referred to as stage 1 and stage 2 of non-rapid eye movement (NREM) sleep); (iv) when the user is in a deep sleep (also referred to as slow-wave sleep, SWS, or stage 3 of NREM sleep); (v) when the user is in rapid eye movement (REM) sleep; (vi) when the user is periodically awake between light sleep, deep sleep, or REM sleep; or (vii) when the user wakes up and does not fall back asleep. The sleep session may also be referred to as a therapy session, or may comprise a therapy session, which can be understood to be the period of time within the sleep session during which the individual is engaged in respiratory therapy (e.g., the use of a respiratory therapy system).

The sleep session is generally defined as ending once the user removes the user interface 120, turns off the respiratory therapy device 110, and gets out of bed. In some implementations, the sleep session can include additional periods of time, or can be limited to only some of the above-disclosed time periods. For example, the sleep session can be defined to encompass a period of time beginning when the respiratory therapy device 110 begins supplying the pressurized air to the airway or the user, ending when the respiratory therapy device 110 stops supplying the pressurized air to the airway of the user, and including some or all of the time points in between, when the user is asleep or awake.

FIG. 3 illustrates an exemplary timeline 300 for a sleep session. The timeline 300 includes an enter bed time (t_{bed}), a go-to-sleep time (t_{GTS}), an initial sleep time (tₛₗₑₑₚ), a first micro-awakening MA₁, a second micro-awakening MA₂, an awakening A, a wake-up time (t_{wake}), and a rising time (tᵣᵢₛₑ).

The enter bed time t_{bed} is associated with the time that the user initially enters the bed (e.g., bed 40 in FIG. 2) prior to falling asleep (e.g., when the user lies down or sits in the bed). The enter bed time t_{bed} can be identified based at least in part on a bed threshold duration to distinguish between times when the user enters the bed for sleep and when the user enters the bed for other reasons (e.g., to watch TV). For example, the bed threshold duration can be at least about 10 minutes, at least about 20 minutes, at least about 30 minutes, at least about 45 minutes, at least about 1 hour, at least about 2 hours, etc. While the enter bed time t_{bed} is described herein in reference to a bed, more generally, the enter time t_{bed} can refer to the time the user initially enters any location for sleeping (e.g., a couch, a chair, a sleeping bag, etc.).

The go-to-sleep time (t_{GTS}) is associated with the time that the user initially attempts to fall asleep after entering the bed (t_{bed}). For example, after entering the bed, the user may engage in one or more activities to wind down prior to trying to sleep (e.g., reading, watching TV, listening to music, using the user device 260, etc.). The initial sleep time (tₛₗₑₑₚ) is the time that the user initially falls asleep. For example, the initial sleep time (tₛₗₑₑₚ) can be the time that the user initially enters the first non-REM sleep stage.

The wake-up time t_{wake} is the time associated with the time when the user wakes up without going back to sleep (e.g., as opposed to the user waking up in the middle of the night and going back to sleep). The user may experience one of more unconscious microawakenings (e.g., microawakenings MA₁ and MA₂) having a short duration (e.g., 5 seconds, 10 seconds, 30 seconds, 1 minute, etc.) after initially falling asleep. In contrast to the wake-up time t_{wake}, the user goes back to sleep after each of the microawakenings MA₁ and MA₂. Similarly, the user may have one or more conscious awakenings (e.g., awakening A) after initially falling asleep (e.g., getting up to go to the bathroom, attending to children or pets, sleep walking, etc.). However, the user goes back to sleep after the awakening A. Thus, the wake-up time t_{wake} can be defined, for example, based at least in part on a wake threshold duration (e.g., the user is awake for at least 15 minutes, at least 20 minutes, at least 30 minutes, at least 1 hour, etc.).

Similarly, the rising time tᵣᵢₛₑ is associated with the time when the user exits the bed and stays out of the bed with the intent to end the sleep session (e.g., as opposed to the user getting up during the night to go to the bathroom, to attend to children or pets, sleep walking, etc.). In other words, the rising time tᵣᵢₛₑ is the time when the user last leaves the bed without returning to the bed until a next sleep session (e.g., the following evening). Thus, the rising time tᵣᵢₛₑ can be defined, for example, based at least in part on a rise threshold duration (e.g., the user has left the bed for at least 15 minutes, at least 20 minutes, at least 30 minutes, at least 1 hour, etc.). The enter bed time t_{bed} time for a second, subsequent sleep session can also be defined based at least in part on a rise threshold duration (e.g., the user has left the bed for at least 4 hours, at least 6 hours, at least 8 hours, at least 12 hours, etc.).

As described above, the user may wake up and get out of bed one more times during the night between the initial t_{bed} and the final tᵣᵢₛₑ. In some implementations, the final wake-up time t_{wake} and/or the final rising time tᵣᵢₛₑ that are identified or determined based at least in part on a predetermined threshold duration of time subsequent to an event (e.g., falling asleep or leaving the bed). Such a threshold duration can be customized for the user. For a standard user which goes to bed in the evening, then wakes up and goes out of bed in the morning any period (between the user waking up (t_{wake}) or raising up (tᵣᵢₛₑ), and the user either going to bed (t_{bed}), going to sleep (t_{GTS}) or falling asleep (tₛₗₑₑₚ) of between about 12 and about 18 hours can be used. For users that spend longer periods of time in bed, shorter threshold periods may be used (e.g., between about 8 hours and about 14 hours). The threshold period may be initially selected and/or later adjusted based at least in part on the system monitoring the user's sleep behavior.

The total time in bed (TIB) is the duration of time between the time enter bed time t_{bed} and the rising time tᵣᵢₛₑ. The total sleep time (TST) is associated with the duration between the initial sleep time and the wake-up time, excluding any conscious or unconscious awakenings and/or micro-awakenings therebetween. Generally, the total sleep time (TST) will be shorter than the total time in bed (TIB) (e.g., one minute short, ten minutes shorter, one hour shorter, etc.). For example, as shown in the timeline 300, the total sleep time (TST) spans between the initial sleep time tₛₗₑₑₚ and the wake-up time t_{wake}, but excludes the duration of the first micro-awakening MA₁, the second micro-awakening MA₂, and the awakening A. As shown, in this example, the total sleep time (TST) is shorter than the total time in bed (TIB).

In some implementations, the total sleep time (TST) can be defined as a persistent total sleep time (PTST). In such implementations, the persistent total sleep time excludes a predetermined initial portion or period of the first non-REM stage (e.g., light sleep stage). For example, the predetermined initial portion can be between about 30 seconds and about 20 minutes, between about 1 minute and about 10 minutes, between about 3 minutes and about 5 minutes, etc. The persistent total sleep time is a measure of sustained sleep, and smooths the sleep-wake hypnogram. For example, when the user is initially falling asleep, the user may be in the first non-REM stage for a very short time (e.g., about 30 seconds), then back into the wakefulness stage for a short period (e.g., one minute), and then goes back to the first non-REM stage. In this example, the persistent total sleep time excludes the first instance (e.g., about 30 seconds) of the first non-REM stage.

In some implementations, the sleep session is defined as starting at the enter bed time (t_{bed}) and ending at the rising time (tᵣᵢₛₑ), i.e., the sleep session is defined as the total time in bed (TIB). In some implementations, a sleep session is defined as starting at the initial sleep time (tₛₗₑₑₚ) and ending at the wake-up time (t_{wake}). In some implementations, the sleep session is defined as the total sleep time (TST). In some implementations, a sleep session is defined as starting at the go-to-sleep time (t_{GTS}) and ending at the wake-up time (t_{wake}). In some implementations, a sleep session is defined as starting at the go-to-sleep time (t_{GTS}) and ending at the rising time (tᵣᵢₛₑ). In some implementations, a sleep session is defined as starting at the enter bed time (t_{bed}) and ending at the wake-up time (t_{wake}). In some implementations, a sleep session is defined as starting at the initial sleep time (tₛₗₑₑₚ) and ending at the rising time (tᵣᵢₛₑ).

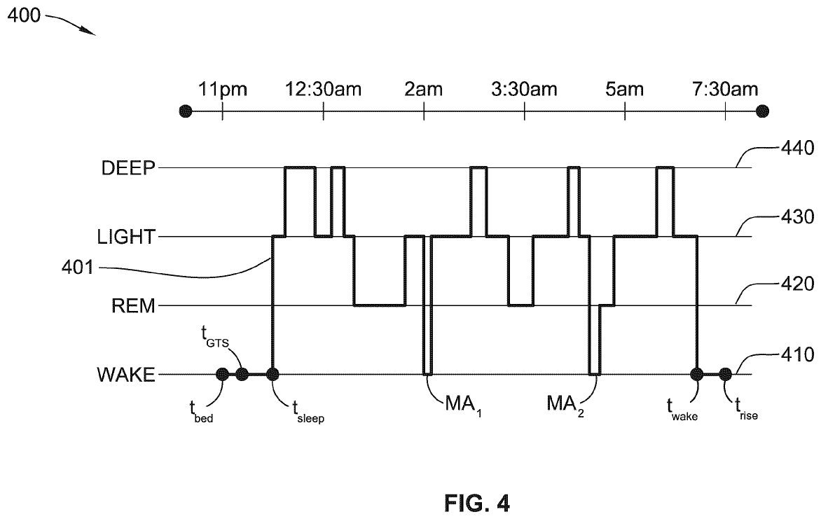

Referring to FIG. 4, an exemplary hypnogram 400 corresponding to the timeline 300 of FIG. 3, according to some implementations, is illustrated. As shown, the hypnogram 400 includes a sleep-wake signal 401, a wakefulness stage axis 410, a REM stage axis 420, a light sleep stage axis 430, and a deep sleep stage axis 440. The intersection between the sleep-wake signal 401 and one of the axes 410-440 is indicative of the sleep stage at any given time during the sleep session.

The sleep-wake signal 401 can be generated based at least in part on physiological data associated with the user (e.g., generated by one or more of the sensors 210 described herein). The sleep-wake signal can be indicative of one or more sleep stages, including wakefulness, relaxed wakefulness, microawakenings, a REM stage, a first non-REM stage, a second non-REM stage, a third non-REM stage, or any combination thereof. In some implementations, one or more of the first non-REM stage, the second non-REM stage, and the third non-REM stage can be grouped together and categorized as a light sleep stage or a deep sleep stage. For example, the light sleep stage can include the first non-REM stage and the deep sleep stage can include the second non-REM stage and the third non-REM stage. While the hypnogram 400 is shown in FIG. 4 as including the light sleep stage axis 430 and the deep sleep stage axis 440, in some implementations, the hypnogram 400 can include an axis for each of the first non-REM stage, the second non-REM stage, and the third non-REM stage. In other implementations, the sleep-wake signal can also be indicative of a respiration signal, a respiration rate, an inspiration amplitude, an expiration amplitude, an inspiration-expiration amplitude ratio, an inspiration-expiration duration ratio, a number of events per hour, a pattern of events, or any combination thereof. Information describing the sleep-wake signal can be stored in the memory device 204.

The hypnogram 400 can be used to determine one or more sleep-related parameters, such as, for example, a sleep onset latency (SOL), wake-after-sleep onset (WASO), a sleep efficiency (SE), a sleep fragmentation index, sleep blocks, or any combination thereof.

The sleep onset latency (SOL) is defined as the time between the go-to-sleep time (t_{GTS}) and the initial sleep time (tₛₗₑₑₚ). In other words, the sleep onset latency is indicative of the time that it took the user to actually fall asleep after initially attempting to fall asleep. In some implementations, the sleep onset latency is defined as a persistent sleep onset latency (PSOL). The persistent sleep onset latency differs from the sleep onset latency in that the persistent sleep onset latency is defined as the duration time between the go-to-sleep time and a predetermined amount of sustained sleep. In some implementations, the predetermined amount of sustained sleep can include, for example, at least 10 minutes of sleep within the second non-REM stage, the third non-REM stage, and/or the REM stage with no more than 2 minutes of wakefulness, the first non-REM stage, and/or movement therebetween. In other words, the persistent sleep onset latency requires up to, for example, 8 minutes of sustained sleep within the second non-REM stage, the third non-REM stage, and/or the REM stage. In other implementations, the predetermined amount of sustained sleep can include at least 10 minutes of sleep within the first non-REM stage, the second non-REM stage, the third non-REM stage, and/or the REM stage subsequent to the initial sleep time. In such implementations, the predetermined amount of sustained sleep can exclude any micro-awakenings (e.g., a ten second micro-awakening does not restart the 10-minute period).

The wake-after-sleep onset (WASO) is associated with the total duration of time that the user is awake between the initial sleep time and the wake-up time. Thus, the wake-after-sleep onset includes short and micro-awakenings during the sleep session (e.g., the micro-awakenings MA₁ and MA₂ shown in FIG. 4), whether conscious or unconscious. In some implementations, the wake-after-sleep onset (WASO) is defined as a persistent wake-after-sleep onset (PWASO) that only includes the total durations of awakenings having a predetermined length (e.g., greater than 10 seconds, greater than 30 seconds, greater than 60 seconds, greater than about 5 minutes, greater than about 10 minutes, etc.)

The sleep efficiency (SE) is determined as a ratio of the total time in bed (TIB) and the total sleep time (TST). For example, if the total time in bed is 8 hours and the total sleep time is 7.5 hours, the sleep efficiency for that sleep session is 93.75%. The sleep efficiency is indicative of the sleep hygiene of the user. For example, if the user enters the bed and spends time engaged in other activities (e.g., watching TV) before sleep, the sleep efficiency will be reduced (e.g., the user is penalized). In some implementations, the sleep efficiency (SE) can be calculated based at least in part on the total time in bed (TIB) and the total time that the user is attempting to sleep. In such implementations, the total time that the user is attempting to sleep is defined as the duration between the go-to-sleep (GTS) time and the rising time described herein. For example, if the total sleep time is 8 hours (e.g., between 11 PM and 7 AM), the go-to-sleep time is 10:45 PM, and the rising time is 7:15 AM, in such implementations, the sleep efficiency parameter is calculated as about 94%.

The fragmentation index is determined based at least in part on the number of awakenings during the sleep session. For example, if the user had two micro-awakenings (e.g., micro-awakening MA₁ and micro-awakening MA₂ shown in FIG. 4), the fragmentation index can be expressed as 2. In some implementations, the fragmentation index is scaled between a predetermined range of integers (e.g., between 0 and 10).

The sleep blocks are associated with a transition between any stage of sleep (e.g., the first non-REM stage, the second non-REM stage, the third non-REM stage, and/or the REM) and the wakefulness stage. The sleep blocks can be calculated at a resolution of, for example, 30 seconds.

In some implementations, the systems and methods described herein can include generating or analyzing a hypnogram including a sleep-wake signal to determine or identify the enter bed time (t_{bed}), the go-to-sleep time (t_{GTS}), the initial sleep time (tₛₗₑₑₚ), one or more first micro-awakenings (e.g., MA₁ and MA₂), the wake-up time (t_{wake}), the rising time (tᵣᵢₛₑ), or any combination thereof based at least in part on the sleep-wake signal of a hypnogram.

In other implementations, one or more of the sensors 210 can be used to determine or identify the enter bed time (t_{bed}), the go-to-sleep time (t_{GTS}), the initial sleep time (tₛₗₑₑₚ), one or more first micro-awakenings (e.g., MA₁ and MA₂), the wake-up time (t_{wake}), the rising time (tᵣᵢₛₑ), or any combination thereof, which in turn define the sleep session. For example, the enter bed time t_{bed} can be determined based at least in part on, for example, data generated by the motion sensor 218, the microphone 220, the camera 232, or any combination thereof. The go-to-sleep time can be determined based at least in part on, for example, data from the motion sensor 218 (e.g., data indicative of no movement by the user), data from the camera 232 (e.g., data indicative of no movement by the user and/or that the user has turned off the lights), data from the microphone 220 (e.g., data indicative of the using turning off a TV), data from the user device 260 (e.g., data indicative of the user no longer using the user device 260), data from the pressure sensor 212 and/or the flow rate sensor 214 (e.g., data indicative of the user turning on the respiratory therapy device 110, data indicative of the user donning the user interface 120, etc.), or any combination thereof.

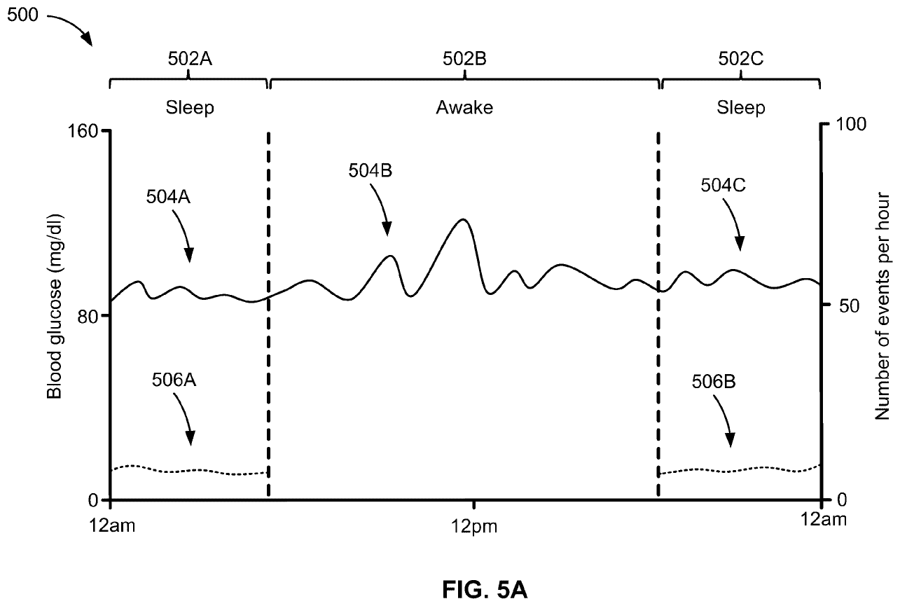

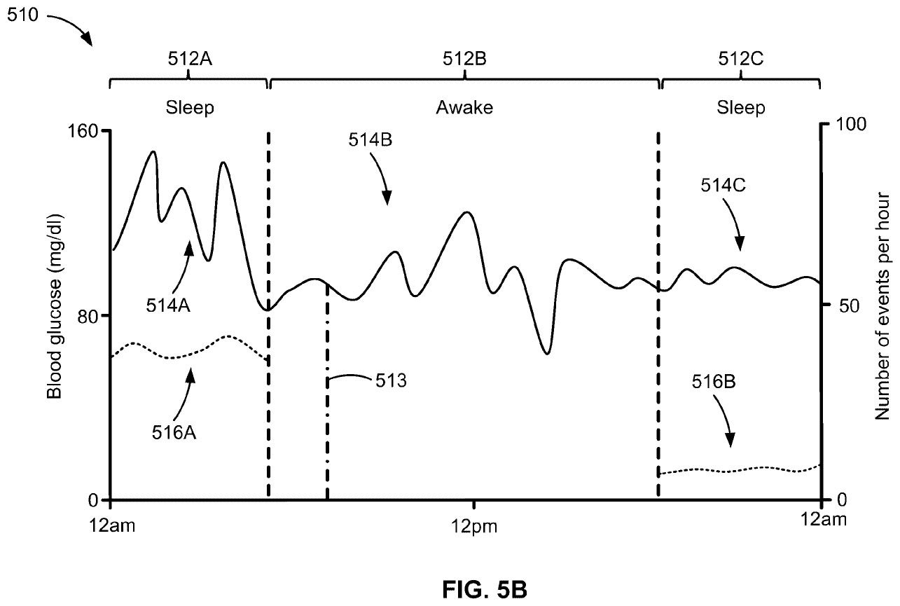

Referring now to FIGS. 5A and 5B, individuals with diabetes who also suffer from SDB (such as OSA or CSA) often must deal with interactions between the two conditions. For example, sleep quality can affect an individual's sensitivity to insulin. Negative impacts on sleep quality due to SDB (such as a sleep session of short duration, a shorter time spent in desirable sleep stages during the sleep session and/or a longer time spent in undesirable sleep stages during the sleep session, an excessive duration of low-quality sleep, etc.) can negatively impact the effectiveness of the diabetes treatment plan that the individual adheres to (e.g., the effectiveness of diabetes, medication, diet, exercise, etc.). Similarly, positive effects on sleep quality due to the use of a respiratory therapy system (such as a sleep session of long duration, a longer time spent in desirable sleep stages during the sleep session and/or a shorter time spent in undesirable sleep stages during the sleep session, a limited duration of low-quality sleep, etc.) can positively impact the effectiveness of the diabetes treatment plan that the individual adheres to (e.g., the effectiveness of diabetes, medication, diet, exercise, etc.). In another example, OSA (or the lack thereof due to the use of a respiratory therapy system) can affect an individual's ability to metabolize glucose. Thus, diabetic individuals who suffer from OSA can often have great difficulties in managing their diabetes. In a further example, the use of a respiratory therapy system to treat SDB can alter the effectiveness of the individual's diabetes treatment plan. A variety of different techniques can be used to aid the individual in managing their diabetes when using a respiratory therapy system intended to treat SDB (or other conditions), and vice-versa. More information can be found at least in paragraphs [0001]-[0028] and FIGS. 1-3 of U.S. App. No. 2009/0007918, which is hereby incorporated by reference herein in its entirety.

FIGS. 5A and 5B illustrate the interaction between a diabetes treatment plan and a respiratory therapy plan. FIG. 5A shows a dual-vertical axis plot 500 of blood glucose levels over a 24-hour period, and the number of events per hour over the same 24-hour period, for an individual who has generally controlled blood glucose levels and SDB. The horizontal axis of the plot is divided into three period of time. The first time period 502A corresponds to at least a portion of a first sleep session. The second time period 502B corresponds to the following day when the individual is awake/not in a sleep session. The third time period 502C corresponds to at least a portion of a second sleep session following the individual being awake. Generally, the individual is asleep for a majority of the first time period 502A and the third time period 502C—which occur during sleep sessions—but the individual could be awake for some amount of time within these time period.

The plot of the individual's blood glucose levels includes a first portion 504A occurring during the first time period 502A, a second portion 504B occurring during the second time period 502B, and a third portion 504C occurring during the third time period 502C. Similarly, the plot of the number of events per hour includes a first portion 506A occurring during the first time period 502A, and a second portion 506B occurring during the third time period 502C. Because the individual is not in a sleep session during the second time period 502B, there is no portion of the plot of the number of events that occurs during the second time period 502B.

As can been in FIG. 5A, the individual's blood glucose levels in the first portion 504A and the third portion 504C remain relatively stable during the first time period 502A and the third time period 502C when the individual is in a sleep session (and is generally asleep). Correspondingly, the individual experiences a relatively low and stable number of events per hour during the first time period 502A and the third time period 502C. Moreover, the individual's blood glucose levels in the second portion 504B are generally controlled during the second time period 502B (e.g. during the day), which in some cases may be due to suitably controlled diet and/or exercise.

FIG. 5B shows a dual-vertical axis plot 510 that is similar to plot 500. However, plot 510 is for an individual whose blood glucose levels and/or OSA is more uncontrolled than the individual in FIG. 5A. Plot 510 is divided into three periods of time. The first time period 512A corresponds to at least a portion of a first sleep session. The second time period 512B corresponds to the day following the first sleep session. The third time period 512C corresponds to at least a portion of a second sleep session. The individual is generally asleep for a majority of the first time period 512A and the third time period 512C during the sleep sessions. The plot of the individual's blood glucose levels includes a first portion 514A during the first time period 512A, a second portion 514B during the second time period 512B, and a third portion 514C during the third time period 512C. The plot of the number of events per hour that the individual experiences during the sleep session includes a first portion 516A occurring during the first time period 512A, and a second portion 516B occurring during the third time period 512C.

As can be seen in FIG. 5B, during the first time period 512A (e.g., during the first sleep session), the individual's blood sugar levels are elevated and unstable, as shown in the first portion 514A. Correspondingly, the individual experiences an increased number of events per hour as shown in the first portion 516A. During the second time period 512B (e.g., during the day following the first sleep session), diabetes medication is administered to the individual at time 513. Then, during the third time period 512C (e.g., during the second sleep session), the individual's blood sugar levels are decreased and/or adequately controlled.

Thus, when the individual's SDB is generally uncontrolled and/or not being treated effectively (potentially leading to poor sleep quality), the individual's blood glucose levels tend to elevated and/or unstable. This poor sleep can also lead to the individual being tired during the day and/or not adhering to their prescribed diet (e.g., by eating sugary foods), which can contribute to elevated and/or unstable blood glucose levels later on if not treated. Described in more detail herein are various methods and techniques for monitoring an individual with diabetes for potential interactions between a diabetes treatment plan and a respiratory therapy plan.

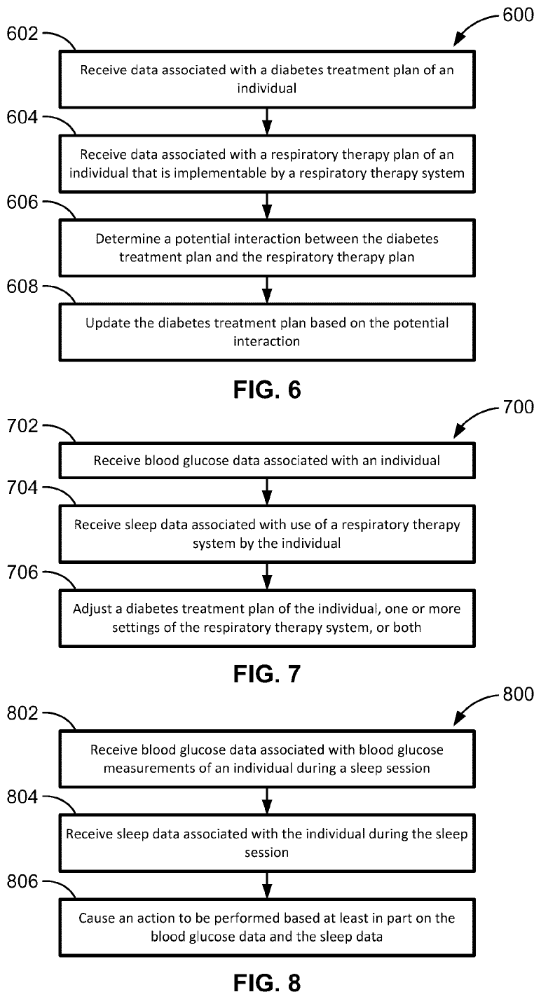

FIG. 6 illustrates a method 600 for monitoring an individual with diabetes, and for updating the individual's diabetes treatment plan in light of any interactions with the individual's use of a respiratory therapy system. Generally, a control system having one or more processors (such as control system 200 of system 10) is configured to implement the steps of method 600. A memory device (such as memory device 204 of system 10) that is coupled to the control system can be used to store machine-readable instructions that are executed by the one or more processors of the control system to implement the steps of method 600. The memory device can also store any type of data utilized in the steps of method 600. In some cases, method 600 can be implemented using a system (such as system 10) that includes a respiratory therapy system (such as respiratory therapy system 100) having a respiratory therapy device configured to supply pressurized air (such as respiratory therapy device 110), a user interface (such as user interface 120) coupled to the respiratory therapy device via the conduit (such as conduit 140). The user interface is configured to engage with the user, and aids in directing the pressurized air to the user's airway. Method 600 can also be implemented using a computer program product (such as a non-transitory computer readable medium) comprising instructions that when executed by a computer, cause the computer to carry out the steps of method 600.

Step 602 of method 600 includes receiving data associated with a diabetes treatment plan of the individual. The received data can be indicative of any characteristic of the diabetes treatment plan, including a diabetes medication plan, a diet plan, an exercise plan, a sleep plan, a blood glucose measurement plan, etc. The data indicative of the diabetes medication plan can include types of diabetes medication that the individual is taking, amounts of the medication that the individual has been prescribed or recommended, a schedule for taking diabetes medication, and other information. The diabetes medication can include any suitable diabetes medication, including insulin, metformin, sulfonylureas, meglitinides, glinides, thiazolidinediones, dipeptidyl peptidase 4 (DPP-4) inhibitors, glucagon-like peptide-1 (GLP-1) receptor agonists, sodium-glucose transport protein 2 (SGLT2) inhibitors, other medicines, or any combination thereof. The diet plan can include, for example, a desired amount of calories per day, a desired amount of macronutrients per day (e.g., the amount of protein, carbohydrates, and fats per day), a meal schedule, desired foods, etc. The exercise plan can include, for example, an exercise schedule, different types of exercises, durations of exercises, etc.

Step 604 of method 600 includes receiving data associated with a respiratory therapy plan of the individual. The respiratory therapy plan can be implemented using a respiratory therapy system (such as the respiratory therapy system 100) that supplies pressurized air to the individual using a respiratory therapy device (such as respiratory therapy device 110), and a user interface (such as user interface 120) coupled to the respiratory therapy device via a conduit (such as conduit 140). In some cases, the respiratory therapy plan is designed to treat the individual's SDB, which could include OSA, CSA, both, or other types and/or combinations of SDB.

The received data can be indicative of a variety of different characteristics of the respiratory therapy plan. The received data can be indicative of the range of different pressure levels that the pressurized air may have (e.g., a minimum pressure, a maximum pressure, an incremental value between different pressure levels, a starting pressure, an ending pressure, etc.), a ramp time of the pressurized air (e.g., how long it takes for the pressure of the air to increase to the desired therapy pressure from the beginning of the use of the respiratory therapy system), the flow rate of the pressurized air, the humidity level of the pressurized air, whether any medicaments are supplied or injected into the individual's airway via the pressurized air, whether and how the respiratory therapy device will operate in different sleep stages of the sleep session (e.g., different operations in a light sleep stage versus a REM sleep stage), and others characteristics. The data associated with the respiratory therapy plan could also include physiological data. This physiological data may be associated with past uses of the respiratory therapy system by the individual according to the respiratory therapy plan, and/or according to other respiratory therapy plans. The physiological data could also be associated with past uses of a respiratory therapy system by other individuals, according to the respiratory therapy plan or different respiratory therapy plans.

Step 606 of method 600 includes determining a potential interaction between the diabetes treatment plan and the respiratory therapy plan. Step 608 of method 600 includes updating the diabetes treatment plan based on the potential interaction. The use of the respiratory therapy system according to the respiratory therapy plan can impact the effectiveness of the diabetes treatment plan in a variety of different. For example, use of the respiratory therapy system (e.g., to treat SBD) may change how the individual's blood glucose levels respond to medication, diet, exercise, etc., such that the diabetes treatment plan less effective at treating the individual's diabetes. The use of the respiratory therapy system could also aid in treating the individual's diabetes, e.g., the diabetes treatment plan is more effective at treating the individual's diabetes when used in conjunction with the respiratory therapy system. In these cases, the diabetes treatment plan may be unnecessarily strict in certain aspects, such as unnecessarily high or frequent doses of medication. An overly strict diabetes treatment plan may also inadvertently reduce compliance, thereby negatively impacting the treatment of the individual's diabetes. By determining a potential interaction between the diabetes treatment plan and the respiratory therapy plan, the diabetes plan can be updated so as to avoid this interaction.

In some implementations, updating the diabetes treatment plan can include updating various aspects related to the individual's usage of diabetes medication, or determining updates to various aspects related to the individual's usage of diabetes medication. The updating can include adjusting the amount of diabetes medication that the individual receives, adjusting the frequency at which the individual receives the diabetes medication, adjusting one or more times of day when the individual receives the diabetes medication, adjusting the type of diabetes medication currently used by the individual, adjusting other aspects related to the individual's diabetes medication, or any combination thereof. In one example, if it is determined that the interaction between the diabetes treatment plan and the respiratory therapy plan will decrease the effectiveness of the individual's diabetes medication, the diabetes plan can be adjusted to counter this decrease in the effectiveness. This adjustment can include increasing the dosage of the diabetes medication that the individual receives, increasing the frequency at which the individual receives the diabetes medication, changing the time of day when the individual receives the diabetes medication, changing which type of diabetes medication the individual receives, and other actions. In some cases of this example, the dosage and/or frequency could be decreased as well.

In another example, the individual's usage of diabetes medication can be adjusted if the respiratory therapy plan will increase the effectiveness of the diabetes medication. This adjustment can include decreasing the dosage of the diabetes medication that the individual receives, decreasing the frequency at which the individual receives the diabetes medication, changing the time of day when the individual receives the diabetes medication, changing which type of diabetes medication the individual receives, and other actions. In some cases of this example, the dosage and/or frequency could be increased as well.

In either example, any aspect related to the individual's diabetes medication that needs to be updated can be updated over time. For example, the dosage of the individual's diabetes medication can initially be adjusted by a small amount in advance of the anticipated interaction between the diabetes treatment plan and the respiratory therapy plan. As the individual continues to use the respiratory therapy system in adherence with the respiratory therapy plan, the dosage can be updated as needed. In these cases, feedback data related to the effectiveness of the modification can be generated and analyzed, so the interaction can be monitored over time, and the modification can be updated.

In some implementations, adjusting the diabetes treatment plan can include adjusting the individual's diet plan and/or the individual's exercise plan, or determining updates to various aspects related to the individual's diet plan and/or the individual's exercise plan. For example, if the interaction between the diabetes treatment plan and the respiratory therapy plan renders the diabetes treatment plan more or less effective, the diet plan could be adjusted by modifying (e.g., increasing or decreasing) the amount of calories the individual consumes and/or the amount of carbohydrates that the individual consumes. In another example, the exercise plan can be adjusted by increasing the amount of exercise if the diabetes treatment plan will be less effective, decreasing the amount of exercise if the diabetes treatment plan will be more effective, or other actions.

In further implementations, stress events during the use of the respiratory therapy system according to the respiratory therapy plan can impact the individual's glycemic control (e.g., the individual's ability to naturally control their blood glucose levels). In these implementations, determining the potential interaction at step 606 can include determining whether such stress events are likely to occur during use of the respiratory therapy system. In some examples, if such stress events are likely to occur, step 608 can include adjusting the diabetes treatment plan in order to counteract the expected reduction in the individual's glycemic control, and/or adjusting the respiratory therapy plan to reduce the occurrence and/or severity of the stress events. In other examples, step 606 may result in a determination that stress events are less likely to occur as the result of future uses of the respiratory therapy system as compared to past uses (for example if the respiratory therapy plan is modified). In these examples, step 608 can include adjusting the diabetes treatment plan to counteract the expected increase in the individual's glycemic control.

In some implementations, the determined interaction includes determining that the individual will experience poor sleep when they initially start using the respiratory therapy system because, for example, the individual may not be used to wearing the user interface during the sleep session. This anticipated poor sleep can impact the effectiveness of the diabetes treatment plan. Thus, in some cases, the diabetes treatment plan can be adjusted in advance of the individual beginning to use the respiratory therapy system to account for the anticipated poor sleep. In other cases, it may be determined that the individual's diabetes treatment plan should stay the same even after the individual initially begins to use the respiratory therapy system, because of the anticipated poor sleep. Thus, the diabetes treatment plan can remain unmodified until the individual's sleep improves, which may come after adjusting the type of user interface that is used, adjusting pressure settings, etc.

The diabetes treatment plan can be considered to exist in a variety of different states. A given state of the diabetes treatment plan can include a specific diabetes medication plan, and/or other plans as well. Whenever some aspect of the diabetes treatment plan is modified or adjusted, the diabetes treatment plan can be considered to be in a different state. For example, a first state may include a diabetes medication plan requiring the individual to take a specific diabetes medication according to a specific schedule. A second state may include a diabetes medication plan requiring the individual to take a different diabetes medication according to the same schedule, the same diabetes medication according to the same schedule, or a different diabetes medication according to a different schedule.

Thus, when the individual's diabetes treatment plan is updated in view of a potential interaction between the diabetes treatment plan and the individual's respiratory therapy plan, this updating can be considered to be transitioning the diabetes treatment plan between different states. In some implementations of method 600, updating the diabetes treatment plan can include transitioning the diabetes treatment plan from a first state to a second state in advance of using the respiratory therapy system in accordance with the respiratory therapy plan. For example, determining the potential interaction can include determining whether an upcoming use of the respiratory therapy system in accordance with the respiratory therapy plan will impact the effects of the individual's diabetes treatment plan. If the diabetes treatment plan currently exists in a first state, the diabetes treatment plan can be updated from that first state to a second state prior to the upcoming use of the respiratory therapy system.

The diabetes treatment plan could be updated to the second state at a variety of different dates/times relative to the upcoming use of the respiratory therapy system. For example, if the upcoming use of the respiratory therapy system is at night, the diabetes treatment plan may be updated to the second state in time for the updated diabetes treatment plan to be in effect for most of or all of the day immediately preceding the use of the respiratory therapy system. The diabetes treatment plan could also be updated such that the diabetes treatment plan is in the second stage only once the upcoming use of the respiratory therapy system ends (e.g., so that the diabetes treatment plan is in the second state starting the day after the initial use of the respiratory therapy system the preceding night).

In some implementations, method 600 is used to anticipate interactions between the individual's diabetes treatment plan and the use of a respiratory therapy system according to a respiratory therapy plan when the individual has never used a respiratory therapy system (or at least never used a respiratory therapy system in accordance with the current respiratory therapy plan). In these implementations, the upcoming use of the respiratory therapy system is the initial (e.g., first) time that the individual will use the respiratory therapy system in accordance with the respiratory therapy plan. Thus, the diabetes management plan can generally be considered to exist in an initial state (e.g., the first state) prior to the individual ever using a respiratory therapy system. Once the individual determines that they will begin using a respiratory therapy system in accordance with a respiratory therapy plan (for example following the advice of a healthcare practitioner), the diabetes treatment plan can be updated to a different state (e.g., the second state) prior to the initial use of the respiratory therapy system. Thus, any potential negative effects of beginning the use of the respiratory therapy system according to the respiratory therapy plan can be avoided by proactively updating the diabetes treatment plan.

In some cases, the different state can be considered to be a final state, e.g., the diabetes treatment plan is updated in advance of the initial use of the respiratory therapy system and then does not need to be updated again. However, in other cases, the different state is only an intermediate state, and it is planned that the diabetes treatment plan will continually be adjusted to different states. For example, the diabetes treatment plan could be updated to the intermediate state (e.g., the second state) prior to the initial use of the respiratory therapy system according to the respiratory therapy plan, and then updated to a final state (e.g., the third state) after the respiratory therapy system has been used for one or more sleep sessions, and the interactions between the diabetes treatment plan and the respiratory therapy plan can be better understood.

In some implementations, method 600 can further including receiving historical data associated with other individuals who have diabetes and have used or will use a respiratory therapy system. The potential interaction between the current individual's diabetes treatment plan and respiratory therapy plan can be determined based at least in part on the historical data. Any updates to the diabetes treatment plan can also be based at least in part on the historical data. The historical data can generally include any data related these other individuals, including age, sex, body mass index (BMI), etc. The historical data will also generally include data associated with the diabetes treatment plans of the other individuals, data associated with the respiratory therapy plans of the other individuals, data associated with interactions between the diabetes treatment plans and the respiratory therapy plans, data associated with changes that were made to the diabetes treatment plans of those other individuals, and other types of data.

The historical data can be analyzed to determine what type of interaction between the individual's diabetes treatment plan and the individual's respiratory therapy plan might be likely. For example, if the historical data includes data associated with an individual that is similar to the current individual, and/or has a similar diabetes treatment plan or respiratory therapy plan, the method 600 can determine that the interaction between the diabetes treatment plan and the respiratory therapy plan of the current individual may be similar to the other individual.

In one example of method 600, using a respiratory therapy system in adherence with a respiratory therapy plan could cause the individual to sleep longer. However, if the individual's sleep sessions last longer, the individual's blood glucose may rise higher toward the end of the sleep sessions, since the individual is asleep for longer and not actively managing their diabetes. Thus, the individual's diabetes treatment plan could be modified to counteract this expected rise in blood glucose levels. In another example, using the respiratory therapy system in adherence with a respiratory therapy plan can cause the individual to sleep less, due to insomnia induced by the use of the respiratory therapy system. The individual may thus have a higher than expected blood glucose level at the end of the sleep session or a lower than expected blood glucose level at the end of the sleep session. A higher than expected blood glucose level may result if the individual's blood glucose was not adequately metabolized during the sleep session. A lower than expected blood glucose level may result if an expected rise in blood glucose toward the end of the sleep session did not occur. In some cases, this expected rise or fall would be evident from historical blood glucose data of the individual. In these cases, the individual's diabetes treatment plan can be modified to counteract this expected rise or fall in blood glucose levels.

While the diabetes treatment plan of the individual can be updated if it is determined that the interaction will result in the diabetes treatment plan being less effective than intended or more effective than intended, updates to the diabetes treatment plan could be made for other reasons as well. For example, determining the potential interaction may reveal that the respiratory therapy plan will be less effective. In this example, the diabetes treatment plan can be updated in a fashion that does not alter the effectiveness of the diabetes treatment plan in treating the individual's diabetes, but does result in the respiratory therapy plan being more effective in treating the individual's SDB. In some cases of this example, it can be determined that the current diabetes treatment plan will make it less likely that the individual adheres to the respiratory therapy plan. Thus, the diabetes treatment plan can be updated in a fashion that will increase the likelihood that the individual will adhere to the respiratory therapy plan.

Interactions between a given diabetes treatment plan and given respiratory therapy plan can be unique to the individual adhering to the plans. Thus, in some cases, the interactions between the plans can be continuously learned and updated as the individual is monitored during their adherence to the plans over a certain time period (e.g., one day, two days, one week, one month, etc.). A potential interaction that is determined at step 606 could thus be based at least in part on past known interactions.

In some cases, there may be periods during a sleep session when the individual does not use the respiratory therapy system, e.g., the individual is “off therapy.” By comparing the sleep data and the blood glucose data (which will both generally be time-stamped), it can be determined how the individual's blood glucose levels (or any other relevant marker) are impacted (positively or negatively) based on the duration and frequency of the use of the respiratory therapy system during the sleep session. In cases where the potential interaction is based at least in part on past known interactions, any update to the individual's diabetes treatment plan can be based on this determination.

FIG. 7 illustrates a method 700 for monitoring an individual with diabetes and determining if the individual's use of a respiratory therapy system is positively or negatively affecting the management of the individual's diabetes. Generally, a control system having one or more processors (such as control system 200 of system 10) is configured to implement the steps of method 700. A memory device (such as memory device 204 of system 10) that is coupled to the control system can be used to store machine-readable instructions that are executed by the one or more processors of the control system to implement the steps of method 700. The memory device can also store any type of data utilized in the steps of method 700. In some cases, method 700 can be implemented using a system (such as system 10) that includes a respiratory therapy system (such as respiratory therapy system 100) having a respiratory therapy device configured to supply pressurized air (such as respiratory therapy device 110), a user interface (such as user interface 120) coupled to the respiratory therapy device via the conduit (such as conduit 140). The user interface is configured to engage with the user, and aids in directing the pressurized air to the user's airway. Method 700 can also be implemented using a computer program product (such as a non-transitory computer readable medium) comprising instructions that when executed by a computer, cause the computer to carry out the steps of method 700.

Step 702 of method 700 includes receiving blood glucose data that is indicative of one or more blood glucose measurements of the individual. The blood glucose data can be obtained in any suitable fashion. In some implementations, the blood glucose data is obtained from a blood glucose meter or system that the individual uses. For example, the individual may use a blood glucose meter to take single blood glucose measurements. In another example, the individual may use a continuous glucose monitor that periodically generates blood glucose measurements. The blood glucose data from these devices can be stored on the meters themselves, in one or more device separate from the user (such as the user device 260, which could be a smart phone, a computer, etc.), in cloud storage, or in other locations. The blood glucose data can generally be retrieved from any location where it is stored for use with method 700.

In some implementations, blood glucose data can be obtained using a sensor positioned within the respiratory therapy system. For example, an analyte sensor (such as analyte sensor 252) could be positioned in the user interface, the conduit, the respiratory therapy device, or any combination thereof. The analyte sensor can detect and measure one or more indicators of the individual's blood glucose in the individual's breath. Such indicators can include, for example, ketones (such as acetone) which are exhaled in the individual's breath.

Generally, the blood glucose data is indicative of recently-obtained blood glucose measurements. The blood glucose measurements could be from the past day, the past week, the past week, etc. The blood glucose measurements could alternatively or additionally be obtained during the sleep session. For example, one or more blood glucose measurements can be obtained at the beginning of the sleep session before the individual falls asleep. In cases where the individual uses a continuous glucose monitor, the blood glucose measurements can be obtained throughout the day and/or night, including during portions of a sleep session when the individual is asleep.

Step 704 of method 700 includes receiving sleep data of the individual that is associated with the individual's use of a respiratory therapy system during one or more prior sleep sessions. The sleep data can be generated by the respiratory therapy system that the individual uses during the sleep session, and/or by other devices that are separate from the respiratory therapy system. For example, a number of different sensors (such as the sensors 210 of system 10) can be used to generate physiological data associated with the user, even if they are not an integrated part of the respiratory therapy system.

The sleep data can comprise different types of data, including data related to the individual's sleep (e.g., sleep metrics such as sleep quality, sleep hygiene, etc.) and data related to the individual's respiratory therapy (e.g., the individual's use and duration of use of a respiratory therapy system). For example, the sleep data can include a time spent asleep during the one or more prior sleep sessions, a time spent awake during the one or more prior sleep sessions, a time spent in each of one or more sleep stages (e.g., a light sleep stage, a deep sleep stage, a REM sleep stage) during the one or more prior sleep sessions, a number of events experienced during the one or more prior sleep sessions, a type of each event (e.g., snoring, apneas, central apneas, obstructive apneas, mixed apneas, hypopneas, RERAs, flow limitations, mask leaks, etc.) experienced during the one or more prior sleep sessions, pressure data associated with the pressure of the pressurized air supplied by the respiratory therapy system during the one or more prior sleep sessions, flow data associated with the flow of the pressurized air supplied by the respiratory therapy system during the one or more prior sleep sessions, physiological data associated with the user, other types of data, or any combination thereof.

Step 706 of method 700 includes adjusting the individual's diabetes treatment plan, respiratory therapy plan, or both, based at least in part on the blood glucose data and the sleep data. Step 706 can include analyzing the blood glucose data and/or the sleep data to see if the individual's use of the respiratory therapy system has impacted the effectiveness of the individual's diabetes treatment plan in treating the individual's diabetes, or to see if the individual's adherence to their diabetes treatment plan has impacted their sleep or the effectiveness of the respiratory therapy system.

As noted previously, in some cases there may be periods during a sleep session when the individual does not use the respiratory therapy system, e.g., the individual is “off therapy.” By comparing the sleep data and the blood glucose data it can be determined how the individual's blood glucose levels are impacted (positively or negatively) based on the duration and frequency of the use of the respiratory therapy system during the sleep session. The individual's diabetes treatment plan and/or respiratory therapy plan can then be modified based on this determination.

In some implementations, method 700 can include determining from the blood glucose data that the individual has experienced an increased blood glucose level during one or more of the prior sleep sessions, and/or during the day(s) following the one or more prior sleep sessions. The increased blood glucose level can be defined in any suitable fashion. In some cases, it is determined that individual has experienced an increased blood glucose level if their average blood glucose level during some time period (e.g., during a sleep session and/or during all or part of the 24 hours following the sleep session) is greater than or equal to a threshold value. In other cases, an increased blood glucose level is indicated if a threshold number of individual blood glucose measurements is greater than or equal to a threshold value. In further cases, an increased blood glucose level is indicated if a single blood glucose measurement is greater than or equal to a threshold value.

In any of these cases, the individual's diabetes treatment plan can be adjusted (or adjustments to the individual's diabetes treatment plan can be determined) in order to better control (e.g., decrease blood glucose levels, prevent blood glucose spikes, etc.) the individual's blood glucose levels (e.g., blood glucose levels during future sleep sessions, during a future portion(s) of the current sleep session, and/or during the day or days following the future sleep sessions). This adjustment could include any adjustment discussed herein, including increasing the amount of diabetes medication received by the individual, increasing the frequency at which the individual receives their diabetes medication, adjusting the time at which the individual receives their diabetes medication, other adjustments, or any combination thereof. After making adjustments made to the individual's diabetes treatment plan (such as adjustments to the individual's diabetes medication), the individual can be monitored over a time period (e.g., one day, one week, etc.) to determine the impact of the adjustment. The monitoring can include analyzing sleep data associated with one or more sleep sessions and blood glucose data over a time period that includes at least the one or more sleep sessions to determine the impact of the adjustment. Further adjustments can then be made.

Additionally or alternatively, the settings of the respiratory therapy system can be modified (or modifications to the settings of the respiratory therapy system can be determined) to better control (e.g., decrease blood glucose levels, prevent blood glucose spikes, etc.) the individual's blood glucose levels (e.g., blood glucose levels during future sleep sessions, during a future portion(s) of the current sleep session, and/or during the day or days following the future sleep sessions). The modification of the respiratory therapy system settings can be used to modify the intended therapy effect of the respiratory therapy system and/or to modify the effect(s) of the use of the respiratory therapy system on the individual's sleep (e.g., sleep quality), and can include adjusting the pressure of the pressurized air delivered by the respiratory therapy system, adjusting the flow rate of the pressurized air, adjusting the ramp time of the respiratory therapy system, adjusting other settings, or any combination thereof.

In some cases, the modifications to the settings increase the intended therapy effect of the respiratory therapy system, which can result in decreasing the number of events that the individual experiences during the future sleep sessions, reducing the severity of the events that the individual experiences during the future sleep session, and other effects. In certain cases, the settings of the respiratory therapy system could also be modified in order to decrease the intended therapy effect of the respiratory therapy system.

In some cases, the modifications to the settings of the respiratory therapy system increase compliance with the respiratory therapy plan and the respiratory therapy system. As discussed herein with respect to FIGS. 5A and 5B, use of the respiratory therapy system can reduce events experienced during the sleep session, and improve the individual's glycemic control. Compliance with the respiratory treatment plan (e.g., continued use of the respiratory therapy system) can thus improve glycemic control. Various modifications can be done to increase compliance. For example, the pressure of the pressurized air supplied by the respiratory therapy system can be modified to decrease the chances that the individual will stop using the respiratory therapy system. The type of user interface that the individual uses could also be modified if the current user interface type is irritating, and causing the individual to stop using the respiratory therapy system. Positional-based events could also be detected, for example by using data from the motion sensor 218 and/or other sensors. If it is determined that the individual is experiencing more events in a certain position (e.g., when sleeping on their back), the respiratory therapy system could be modified to increase air pressure when the individual is in that position. Steps could also be taken to encourage the individual to sleep in other positions, such as on their side.

In some cases, the modifications to the settings of the respiratory therapy system can be done in order to increase the amount of time that the individual will spend asleep and/or will spend in a given sleep stage(s). The sleep stage could be a specific sleep stage (e.g., light sleep stage, deep sleep stage, REM sleep stage), or generally a stage where the user is asleep. For example, modifying the settings of the respiratory therapy system can include limiting the pressures of the pressurized air that can be used in response to the individual experiencing events, in order to decrease the likelihood that the individual will wake up.

In some implementations, the same or similar adjustments to the user's diabetes treatment plan and respiratory therapy plan can be determined and/or made if it is determined that the individual experienced a decreased blood glucose level during one or more prior sleep sessions, and/or during the day(s) following the one or more prior sleep sessions. The decreased blood glucose level can be defined in any suitable fashion, similar to the increased blood glucose level. In some cases, it is determined that individual has experienced a decreased blood glucose level if their average blood glucose level during some time period (e.g., during a sleep session and/or during all or part of the 24 hours following the sleep session) is less than or equal to a threshold value. In other cases, an increased blood glucose level is indicated if a threshold number of individual blood glucose measurements is less than or equal to a threshold value. In further cases, an increased blood glucose level is indicated if a single blood glucose measurement is less than or equal to a threshold value.

In some cases, the adjustment of the settings of the respiratory therapy system can include a change between different modes of operation. As discussed herein, the respiratory therapy system can be used as different types of systems. In a first mode of operation, the respiratory therapy system can be operated as a CPAP system. In a second mode of operation, the respiratory therapy system can be operated as a APAP system. In a third mode of operation, the respiratory therapy system can be operated as a BPAP or VPAP system. In other modes of operation, the respiratory therapy system may be operated as different types of system as well. The blood glucose data and/or the sleep data may reveal that the current mode of operation of the respiratory therapy system is not currently controlling the individual's SDB. The blood glucose data and/or the sleep data may also reveal that the current mode of operation of the respiratory therapy system is currently controlling the individual's SDB, but that the individuals' blood glucose levels are not sufficiently controlled. In either case, the mode of operation of the respiratory therapy system can be changed (e.g., from a CPAP system to an APAP system) in order to better manage the individual's SDB and/or blood glucose levels. In some cases, a balance must be achieved between managing the individual's SDB and managing the individual's blood glucose levels. In these cases, this balance can be learned over time.

In any of these cases, the adjustments to the individual's diabetes treatment plan can be used to better control (e.g., increase blood glucose levels, prevents blood glucose spikes, etc.) the individual's blood glucose levels (e.g., blood glucose levels during future sleep sessions, during a future portion(s) of the current sleep session, and/or during the day or days following the future sleep sessions). This adjustment could include any adjustment discussed herein, including decreasing the amount of diabetes medication received by the individual, decreasing the frequency at which the individual receives their diabetes medication, adjusting the time at which the individual receives their diabetes medication, other adjustments, or any combination thereof. In some implementations, the diabetes treatment plan can be modified to account for stress events (or a lack thereof) experienced by the individual during use of the respiratory therapy system. As noted herein, stress events can impact the individual's glycemic control. The sleep data received in step 704 can include data representative of the individual's sympathetic nervous activation during one or more uses of the respiratory therapy system. Step 706 can then include adjusting the diabetes treatment plan as needed. In some cases, the sleep data can indicate that the individual's glycemic control has been negatively impacted by the stress events caused by the use of the respiratory therapy system. In these cases, the diabetes treatment plan is modified to increase the impact of the diabetes treatment plan on the individual's glycemic control. In other cases, the sleep data can indicate that the individual is experiencing fewer stress events than expected and that the individual's glycemic control has not been as negatively impacted by the use of the respiratory therapy system as expected. In these cases, the diabetes treatment plan is modified to decrease the impact of the diabetes treatment plan on the individual's glycemic control.

The settings of the respiratory therapy system used by the individual can also be modified, for example to modify the intended therapy effect of the respiratory therapy system (e.g., increasing or decreasing the intended therapy effect) and/or to modify the effect(s) of the use of the respiratory therapy system on the individual's sleep (e.g., sleep quality). The modification can include adjusting the pressure of the pressurized air delivered by the respiratory therapy system, adjusting the flow rate of the pressurized air, adjusting the ramp time of the respiratory therapy system, adjusting other settings, or any combination thereof. In an example, an individual may experience nocturnal hypoglycemia if using insulin to treat diabetes. An alarm associated with the respiratory therapy system (such as an alarm implemented by speaker 222) could be activated to wake the individual up, and help mitigate hypoglycemia events.

In some implementations, method 700 further includes analyzing the sleep data to identify one or more sleep stages of the individual during the one or more prior sleep sessions, and analyzing the blood glucose data to determine the blood glucose level of the individual during the identified sleep stages. The adjustment of the diabetes treatment plan and/or any settings of the respiratory therapy system can be based at least in part on the blood glucose level of the individual during the identified sleep stages.

In some implementations, the settings of the respiratory therapy system can be modified to reduce stress events that occur during use of the respiratory therapy system. If the sleep data indicates that the individual is experiencing an increased number and/or severity of stress events, step 706 can include adjusting the settings of the respiratory therapy system to reduce the occurrence and/or severity of such stress events.

In some cases, adjustments can be made and/or determined to the diabetes treatment plan and/or the settings of the respiratory therapy system if the individual is experiencing increased or decreased blood glucose levels during one type of sleep stage, but not a different type of sleep stage. For example, the diabetes treatment plan can be modified if the analysis of the sleep data and the blood glucose data reveals that the individual is experiencing elevated blood glucose levels during REM sleep stages. The modification can include increasing or decreasing the amount of a diabetes medication received by the individual, increasing or decreasing the frequency at which the individual receives the diabetes medication, adjusting the time at which the individual receives the diabetes medication, other actions, or any combination thereof. The settings of the respiratory therapy system could also be adjusted, for example by modifying the pressure or flow rate of the pressurized air, and/or modifying the ramp time of the respiratory therapy system.

Similar to method 600, the diabetes medication can include any suitable diabetes medication, including insulin, metformin, sulfonylureas, meglitinides, glinides, thiazolidinediones, dipeptidyl peptidase 4 (DPP-4) inhibitors, glucagon-like peptide-1 (GLP-1) receptor agonists, sodium-glucose transport protein 2 (SGLT2) inhibitors, other medicines, or any combination thereof.

In some implementations, method 700 includes analyzing sleep data and blood glucose data to determine a blood glucose level of the individual that is associated with events experienced during the sleep session. The modification of the individual's diabetes treatment plan or the settings of the respiratory therapy system can be based in part on how the individual's blood glucose levels respond to events experienced by the individual. For example, if events cause the individual to experience abnormal (e.g., increased or decreased) blood glucose levels after the event(s), the settings of the respiratory therapy system can be modified to decrease the severity of the events and/or decrease the likelihood of the events occurring in the future. These decreases in severity in turn can reduce or eliminate the abnormal (e.g., increased or decreased) blood glucose levels following the event(s). Such modifications could include increasing the pressure of the pressurized during the events and/or prior to the events (or future events), in order to more quickly end the events.

Generally, if an event causes an increased blood glucose level, the increased blood glucose level does not actually occur until some amount of time after the event. Thus, the blood glucose level of the individual that is associated with the event may be the blood glucose level of the individual 10 seconds after the event, 30 seconds events the event, 1 minute after the event, 2minutes after the event, 5 minutes after the event, 10 minutes after the event, etc. However, in some cases, the blood glucose level of the individual that is associated with the event can be the blood glucose level of the individual while the event is occurring.

In some cases, in order to determine if the individual's blood glucose levels associated with the event are increased, the individual's blood glucose levels prior to any events occurring (and a sufficiently long amount of time after the occurrence of any other events) can be used to establish a baseline blood glucose level. The blood glucose level of the individual associated with an event can then be compared to the baseline blood glucose level. In one example, the baseline blood glucose level is the baseline blood glucose level for the entire sleep session, and thus could be the running average blood glucose level of the individual during the sleep session that is determined from blood glucose measurements obtained during the sleep session that would not have been affected by any prior events. In another example, the baseline blood glucose level is the baseline blood glucose level for when the individual is asleep during the sleep session, and thus could be the running average blood glucose level of the individual during the sleep session that is determined from blood glucose measurements obtained while the individual is asleep during the sleep session that would not have been affected by any prior events. In a further example, the baseline blood glucose level is the baseline blood glucose level for when the individual is in the sleep stage that the individual was in when the event in question occurred, and thus could be the running average blood glucose level of the individual during the sleep session that is determined from blood glucose measurements obtained while the individual is in that sleep stage that would not have been affected by any prior events.

In some implementations, method 700 is implemented outside of a sleep session. For example, after analyzing the blood glucose data and the sleep data, settings of the respiratory therapy system can be adjusted, so that the next time the individual uses the respiratory therapy system during a sleep session, the individual will use the updated settings. However, in some cases, method 700 may be implemented during a sleep session. In these implementations, the settings of the respiratory therapy system are updated in real-time as the individual uses the respiratory therapy system during the sleep session.

FIG. 8 illustrates a method 800 for monitoring an individual with diabetes during a sleep session. Generally, a control system having one or more processors (such as control system 200 of system 10) is configured to implement the steps of method 800. A memory device (such as memory device 204 of system 10) that is coupled to the control system can be used to store machine-readable instructions that are executed by the one or more processors of the control system to implement the steps of method 800. The memory device can also store any type of data utilized in the steps of method 800. In some cases, method 800 can be implemented using a system (such as system 10) that includes a respiratory therapy system (such as respiratory therapy system 100) having a respiratory therapy device configured to supply pressurized air (such as respiratory therapy device 110), a user interface (such as user interface 120) coupled to the respiratory therapy device via the conduit (such as conduit 140). The user interface is configured to engage with the user, and aids in directing the pressurized air to the user's airway. Method 800 can also be implemented using a computer program product (such as a non-transitory computer readable medium) comprising instructions that when executed by a computer, cause the computer to carry out the steps of method 800.

Step 802 of method 800 includes receiving blood glucose data that is indicative of one or more blood glucose measurements of the individual taken during the sleep session. Step 802 is generally the same as or similar to step 702 of method 700, and can include obtaining blood glucose data in any suitable fashion. However, the blood glucose data received in step 802 is generally received during the sleep session, and is generally indicative of blood glucose measurements that were made during the sleep session.

Step 804 of method 800 includes receiving sleep data that is associated with the individual during the sleep session. Step 804 is generally the same as or similar to step 704 of method 700. However, similar to step 802, the sleep data received in step 804 is generally received during the sleep session, and is indicative of various different aspects of the sleep session that the individual is currently in (and can include sleep quality data, respiratory therapy data, etc.). The sleep data can include data indicative of the current length of the sleep session (e.g., how long the individual has been in the sleep session), whether the individual is currently awake or asleep, the sleep stage that the individual is currently in (e.g., light sleep stage, deep sleep stage, REM sleep stage, wake stage, etc.), a history of the sleep stages that the individual has been in during the sleep session (including a number and duration of each type of sleep stage), an enter bed time, a go-to-sleep time, an initial sleep time, a wake-up time, a rising time, or characteristics of the sleep session, or any combination thereof.

Step 806 of method 800 includes causing an action to be performed, based at least in part on the received blood glucose data and the received sleep data. Generally, this action will occur if the received data indicates that some unwanted event is occurring during the sleep session, and the action will take place during the sleep session in an attempt to mitigate the unwanted event. In some implementations, the received data may indicate that the individual is experiencing an increased or decreased blood glucose level during the sleep session. In these implementations, the action can include causing the individual to receive some amount of a diabetes medication. For example, if the blood glucose data indicates that the individual is experiencing an increased blood glucose level, the individual can be given medication intended to decrease their blood glucose levels. Similarly, if the blood glucose data indicates that the individual is experiencing a decreased blood glucose level, the individual can be given medication intended to increase their blood glucose levels. The medication can be delivered using a device such as an insulin pump, which could be implantable in the individual's body. The medication could also be delivered via a pill, in which cause the individual would generally be woken up first, and then administered the medication.

In some cases, the blood glucose data and the sleep data may indicate that the individual is experiencing more events when in a certain position during the sleep session (e.g., on their back), leading to elevated blood glucose levels or other unwanted effects. In these cases, step 806 can include encouraging the individual to sleep in a different position (e.g., on their side), for example by sending the individual a recommendation. If the individual has an adjustable mattress with different orientations (e.g., head inclined or declined, feet inclined or declined, etc.), step 806 could additionally or alternatively include adjusting the orientation of the mattress. The mattress orientation can be adjusted so that the individual is caused to lie in a different position where the individual will likely experience fewer events.

In some implementations where the individual has an increased or decreased blood glucose level, method 800 includes determining what sleep stage the individual is currently in, and then causing the individual to receive an amount of diabetes medication when the individual is in one sleep stage but not a different sleep stage. For example, the delivery of diabetes medication during a sleep session could cause the individual to wake up. It is generally less impactful if the individual wakes up when in a light sleep stage as compared to a deep sleep stage or a REM sleep stage, and thus the diabetes medication may be delivered when the individual is in a light sleep stage (or a wake stage), but not in a deep sleep stage or a REM sleep stage. However, it could also be determined that the individual is less likely to wake up if the diabetes medication is delivered during a deep sleep stage and/or a REM sleep stage, as compared to a light sleep stage. Thus, the diabetes medication could be delivered only if the individual is in a deep sleep stage and/or a REM sleep stage, but not if the individual is in a light sleep stage. In some implementations, the action can additionally or alternatively include adjusting settings of the respiratory therapy system in an attempt to mitigate the increased or decreased blood glucose levels.

In some implementations, different actions to combat an increased or decreased blood glucose level can be taken depending on the sleep stage that the individual is currently in. For example, the individual may be more likely to wake up if diabetes medication is delivered when in a light sleep stage as compared to a deep sleep stage or a REM sleep stage. Thus, if an increased or decreased blood glucose level is detected when the user is in a light sleep stage, diabetes medication can be delivered. However, if an increased or decreased blood glucose level is detected when the user is in a deep sleep stage or a REM sleep stage, the settings of the respiratory therapy system can be adjusted in order to mitigate the increased or decreased blood glucose level.

Similar to method 600 and method 700, the diabetes medication can include any suitable diabetes medication, including insulin, metformin, sulfonylureas, meglitinides, glinides, thiazolidinediones, dipeptidyl peptidase 4 (DPP-4) inhibitors, glucagon-like peptide-1 (GLP-1) receptor agonists, sodium-glucose transport protein 2 (SGLT2) inhibitors, other medicines, or any combination thereof.

Blood glucose data received during the sleep session is generated using some sort of automated system that does not require any input from the individual. For example, a continuous glucose meter can be used to measure the individual's blood glucose during the sleep session and generate the blood glucose data. In some implementations, the blood glucose data may not be received until after the sleep session, but is still indicated of blood glucose measurements made during the sleep session, in which case a system that does not require input from the individual will be used. In other implementations, the blood glucose data may be associated with the sleep session, but is indicative of blood glucose measurements made while the user is awake (whether during the sleep session or not). In these implementations, a system that requires input from the individual (such as a blood glucose meter) can be used to generate the blood glucose measurements.

In some implementations, the blood glucose data and the sleep data are received in real-time during the sleep session. In these implementations, the received data can be analyzed in real-time to determine if any immediate action needs to be taken during the sleep session. In other implementations, the blood glucose data and the sleep data can be received during and/or after the sleep session, but are only analyzed after the sleep session.

In some implementations, the action performed in step 806 is performed in real-time during a sleep session. The blood glucose data and the sleep data can be analyzed, and a variety of different actions can be taken to aid any negative occurrences that may be happening during the sleep session (e.g., adjusting settings of the respiratory therapy system, delivering diabetes medication to the individual, etc.). In other implementations, the action performed in step 806 takes place after the sleep session is over. For example, the action could include updating the individual's diabetes treatment plan starting the day after the sleep session, and/or updating settings of the respiratory therapy system starting with the next sleep session.

In further implementations, the action performed at step 806 could be performed during or after the sleep session. For example, the action could include generating a recommendation for the individual based on the blood glucose data and the sleep data. The recommendation could be a recommendation to adjust the individual's diabetes treatment plan, a recommendation to adjust one or more settings of the respiratory therapy system for the next sleep session, a recommendation to consult with a healthcare practitioner, etc. The action could also include notifying a 3rd party (such as a family member, a caretaker, a healthcare practitioner, etc.) of any some negative event or occurrence revealed by the blood glucose data and the sleep data. These actions could take place during or after a sleep session.

In some implementations, the action at step 806 includes delivering diabetes medication to the individual. This delivery could take place in any suitable fashion. For example, in some cases, the individual could have a medication pump (such as an insulin pump) that is configured to deliver medication (such as insulin) to the individual and/or the individual's blood stream, even if the individual is asleep. The action can include operating the medication pump to deliver medication to the individual. In other cases, diabetes medication could be given to the individual using the respiratory therapy system, for example by delivering the diabetes medication into the pressurized air so that the diabetes medication reaches the individual's airway. More information about this

technique can be found at least in paragraphs [0110]-[0196] and FIGS. 5A-12 of WO 2021/084508, which is hereby incorporated by reference herein in its entirety.

One or more elements or aspects or steps, or any portion(s) thereof, from one or more of any of claims 1 to 108 below can be combined with one or more elements or aspects or steps, or any portion(s) thereof, from one or more of any of the other claims 1 to 108 or combinations thereof, to form one or more additional implementations and/or claims of the present disclosure.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations or alternative implementations according to aspects of the present disclosure may combine any number of features from any of the implementations described herein.

1. A method comprising:
receiving data associated with a diabetes treatment plan of the individual;
receiving data associated with a respiratory therapy plan of the individual, the respiratory therapy plan being implementable by a respiratory therapy system during a sleep session;
determining a potential interaction between the diabetes treatment plan of the individual and the respiratory therapy plan of the individual; and
based on the interaction, updating the diabetes treatment plan of the individual and the respiratory therapy plan of the individual.

2. The method of claim 1, wherein updating the diabetes treatment plan includes determining an adjustment to an amount of a diabetes medication to be received by the individual, determining an adjustment to a frequency at which the individual receives the diabetes medication, determining an adjustment to a time at which the individual receives the diabetes medication, or any combination thereof.

3. The method of claim 2, wherein the diabetes medication includes insulin, metformin, sulfonylureas, meglitinides, glinides, thiazolidinediones, dipeptidyl peptidase 4 (DPP-4) inhibitors, glucagon-like peptide-1 (GLP-1) receptor agonists, sodium-glucose transport protein 2 (SGLT2) inhibitors, or any combination thereof.

4. The method of claim 1, wherein the diabetes treatment plan includes a diet plan for the individual, and wherein updating the diabetes treatment plan includes determining an adjustment to the diet plan.

5. The method of claim 1, wherein the diabetes treatment plan includes an exercise plan for the individual, and wherein updating the diabetes treatment plan includes determining an adjustment to the exercise plan.

6. The method of claim 1, wherein updating the diabetes treatment plan includes updating the diabetes treatment plan from a first state to a second state prior to an upcoming use of the respiratory therapy system according to the respiratory therapy plan.

7. (canceled)

8. The method of claim 6, wherein updating the diabetes treatment plan includes updating the diabetes treatment plan to the second state on a date of the upcoming use of the respiratory therapy system.

9. The method of claim 8, wherein updating the diabetes treatment plan includes updating the diabetes treatment plan from the second state to a third state on a later date of a subsequent use of the respiratory therapy system.

10. The method of claim 6, wherein the upcoming use of the respiratory therapy system is an initial use of the respiratory therapy system.

11. The method of claim 10, wherein the first state is an initial state and wherein the second state is an intermediate state or a final state.

12. (canceled)

13. The method of claim 1, further comprising receiving historical data associated with one or more individuals with diabetes, the historical data being indicative of an interaction between a diabetes treatment plan of each of the individuals, and a respiratory therapy plan of each of the individuals.

14. The method of claim 13, wherein determining the potential interaction between the diabetes treatment plan of the individual and the respiratory therapy plan of the individual is based at least in part on the historical data.

15. The method of claim 13, wherein the historical data includes an age of the one or more individuals with diabetes, a sex of the one or more individuals with diabetes, a body mass index (BMI) of the one or more individuals with diabetes, a diabetes medication plan of the one or more individuals with diabetes, or any combination thereof.

16. The method of claim 1, wherein the respiratory therapy plan is for use in treating sleep-disordered breathing (SDB).

17. The method of claim 16, wherein the SDB includes obstructive sleep apnea (OSA), central sleep apnea (CSA), or both.

18. The method of claim 16, wherein the potential interaction includes an increased control of a blood glucose level of the individual, and wherein updating the diabetes treatment plan of the individual includes determining a reduction to an amount of a diabetes medication to be received by the individual, determining a reduction in a frequency at which the individual receives the diabetes medication, or both.

19-56. (canceled)

57. A system comprising:
a respiratory therapy system including:
a respiratory therapy device configured to supply pressurized air; and
a user interface coupled to the respiratory therapy device via a conduit, the user interface being configured to engage a user and aid in directing the supplied pressurized air to an airway of the user;
a memory device storing machine-readable instructions; and
a control system coupled to the memory device, the control system including one or more processors configured to execute the machine-readable instructions to:
receive data associated with a diabetes treatment plan of the individual;
receive data associated with a respiratory therapy plan of the individual, the respiratory therapy plan being implementable by a respiratory therapy system during a sleep session;
determine a potential interaction between the diabetes treatment plan of the individual and the respiratory therapy plan of the individual; and
based on the interaction, update the diabetes treatment plan of the individual.

58-108. (canceled)

109. The method of claim 1, wherein updating the respiratory therapy plan includes a change to the mode of operation of the respiratory therapy system.

110. The method of claim 1, wherein updating the respiratory therapy plan includes a modification to the settings of the respiratory therapy system.

111. The method of claim 110, wherein the modification to the settings of the respiratory therapy system modify the intended therapy effect of the respiratory therapy system and/or modify the effect of the use of the respiratory therapy system on the individual's sleep.

112. The method of claim 110, wherein the modification to the settings of the respiratory therapy system includes adjusting the pressure of the pressurized air delivered by the respiratory therapy system, adjusting the flow rate of the pressurized air, adjusting the ramp time of the respiratory therapy system, or any combination thereof.

113. The method of claim 1, wherein the diabetes treatment plan includes data on a type of diabetes medication, amounts of the diabetes medication prescribed or recommended to the individual, and a schedule for taking the diabetes medication, and wherein the diabetes medication comprises glucagon-like peptide-1 (GLP-1) receptor agonists.
